(19) United States
(12) Patent Application Publication
(10) Pub. No.: US 2003/0134342 A1
(43) Pub. Date: Jul. 17, 2003

(54) Binding-specific peptides, binding-specific carrier molecules, and uses thereof

(76) Inventor: Harvey D. Preisler, Chicago, IL (US)

Correspondence Address: FOLEY & LARDNER, 150 EAST GILMAN STREET, P.O. BOX 1497, MADISON, WI, 53701-1497, US

(21) Appl. No.: 10/062,587

(22) Filed: Jan. 31, 2002

Related U.S. Application Data
(60) Provisional application No. 60/278,465, filed on Mar. 24, 2001.

(51) Int. Cl. G01N 33/574; C07H 21/04; C12P 21/02; C12N 5/06; C07K 16/30

(52) U.S. Cl. 435/7.23; 435/69.1; 435/320.1; 435/326; 530/388.8; 536/23.53

(57) ABSTRACT

The present invention provides peptides that bind cells differentially that are capable of discriminating between normal and malignant cells, and uses of such peptides and specific carrier molecules for screening the binding reactivity of peptides from leukemia cells.

### CLAIM OF PRIORITY

This application claims priority from U.S. Provisional Patent Application No. 60/278,465, filed Mar. 24, 2001, the contents of which are hereby incorporated by reference.

### FIELD OF THE INVENTION

The present invention provides materials and methods using peptides that bind cells. In particular the present invention provides peptides that bind to malignant eukaryotic cells, including leukemic cells.

### BACKGROUND OF THE INVENTION

In normal hematopoeisis, pluripotent stem cells in the bone marrow proliferate (they are capable of self-renewal) and differentiate into either myeloid progenitor cells or lymphoid progenitor cells. These progenitor cells retain the capacity for self-renewal, but they are committed to one of those two major hematopoietic lineages. Depending on the types and amounts of cytokines present, the myeloid and lymphoid cells generate various more mature progenitor cells which proliferate, become committed and further differentiate into the various types of blood cells. Lymphoid cells mature into the T and B cells of the immune system. Myeloid stem cells mature into granulocytes, monocytes, platelets, erythrocytes and eosinophils. Among the cytokines that regulate hematopoiesis are granulocyte colony-stimulating factor (G-CSF), macrophage colony-stimulating factor (M-CSF), several interleukins (IL; in particular IL-3 and IL-6), stem cell factor (SCF), granulocyte-macrophage colony-stimulating factor (GM-CSF), erythropoietin (Epo) and thrombopoietin (Tpo).

In leukemia the blood cells exhibit alterations in their patterns of cellular proliferation and differentiation. Microscopic analysis of biological samples can determine the number of mature cells versus immature blood cells or blasts. A relative proliferation of immature blood cells is an indication of the activity of the leukemia.

Further investigation into a patient's biological samples provides information for further classifying the type of leukemia and the stage of the disease. Hematological analysis of the number and types of blood cells in the peripheral blood, bone marrow, cerebrospinal fluid, FAB analysis of the cells, and immunophenotyping all provide data for classification. The classification of the type of leukemia is used in diagnosis, selection of a treatment regimen, and to ascertain the patient's status or prognosis.

Leukemia is classified as either acute or chronic. In acute leukemia, the blood cells remain blasts and fail to mature and carry out their normal functions. The disease progresses quickly as the number of blasts mounts. In chronic leukemia, blast levels are elevated but these abnormal blasts are more mature and can carry out some normal functions. In chronic leukemia, the number of blasts increases less rapidly than in acute leukemia, and the disease progresses more gradually.

Leukemia of the white blood cells is myelogenous leukemia or lymphocytic leukemia. The most common types of leukemia are acute lymphocytic leukemia (ALL), acute myeloid leukemia (AML, or acute non-lymphoctyic leukemia, ANLL), chronic lymphocytic leukemia (CLL), and chronic myeloid leukemia (CML). Another condition, myelodysplastic syndrome is characterized by abnormalities of the myeloid cells, and can precede the development of myelogenous leukemia.

A treatment regimen may have the goal of inducing remission. “Induction therapy” is used to halt progression of the leukemia. “Consolidation therapy” is used at the end of induction to attempt to prolong the remission stage. Prophylactic treatment (“maintenance therapy”) is another sort of post-induction therapy that serves to prolong remission. In certain cases, complete remission is obtainable. Treatments range from chemotherapy to radiation therapy to bone marrow transplantation to biological therapy or various combinations thereof. The multivaried nature of the disease makes monitoring and specific treatment crucial.

Leukemogenesis appears to be an evolutionary process that involves multiple independent and epigenetic events. Some chromosomal aberrations, such as the Philadelphia chromosome (a reciprocal translocation between chromosomes 9 and 22) demonstrate oncogene abnormalities. The Philadelphia chromosome is found in hemoatopoietic cells of virtually all patients with chronic myelogenous leukemia. Emerging research is also reporting alterations in growth-promoting genes, among other genetic variations.

As noted above, many factors affect cellular proliferation and differentiation. Cytokines are proteins that can activate cellular proliferation and/or differentiation, or influence cells in other ways. Many cytokines and growth factors are pluripotent and can lead to cell growth and/or differentiation in a variety of cell types. Cytokines are secreted primarily by white blood cells (leukocytes) or stromal cells in the marrow and spleen and other organs. Cytokines typically stimulate both humoral and cellular immune responses, and activate phagocytic cells.

Interleukins are cytokines liberated from leukocytes that have their primary effect on leukocytes. For example, interleukin 1-α and -β are derived from macrophages and other antigen presenting cells (APCs), and exert their effects on a large variety of cells including APCs and T cells. Interleukin-2 leads to proliferation of B cells and activated T cells. Interleukin-3 (IL-3) is another cytokine that stimulates the proliferation of myelogenous cells and affects their differentiation. IL-3 contributes to the growth of hematopoietic progenitor cells. Interleukins-4 and -13 stimulate B cell proliferation, eosinophil and mast cell growth and function, IgE and Class II major histocompatibility antigen expression on B cells, and inhibition of monokine production. Interleukin-6 stimulates B cell differentiation and proliferation, and is synergistic with interleukin-1 and tumor necrosis factor on T cells. Interleukin-7 leads to T and B lymphopoiesis. Interluekin-10 promotes B cell proliferation and antibody production. Interleukin-11 has synergistic hematopoietic and thrombopoietic effects.

Other growth factors include the class of cytokines referred to as interferons. Type I interferons include interferon-α, -β and -ω. Type II interferons include interferon-γ, or immune interferon. Interferon-α and -β are liberated from macrophages, neutrophils and some somatic cells. These interferons have antiviral effect, induce expression of Class I MHC molecules on somatic cells, and activate natural killer (NK) cells and macrophages. Interferon-γ is liberated from activated T helper cells and NK cells. Interferon-γ induces expression of Class I MHC antigens on all somatic cells, and Class II MHC antigens on antigen presenting cells and somatic cells. Interferon-γ also activates macrophages, neutrophils, NK cells, and promotes cell-mediated immunity.

Other useful cytokines are colony stimulating factors. Colony stimulating factors stimulate the proliferation of specific pluripotent stem cells in the bone marrow. Granulocyte-colony stimulating factor has specific proliferative effects on cells of the granulocyte lineage. Macrophage-colony stimulating factor has effects on macrophage cell types. Granulocyte-macrophage-colony stimulating factor (GM-CSF) affects both classes of lymphoid cells. GM-CSF affects both proliferation and differentiation of myelogenous cells. In particular, GM-CSF stimulates the proliferation of myeloid cells, and patients who do not produce enough of their own GM-CSF have a poor prognosis.

In some leukemia treatment regimens, GM-CSF is administered to a patient in order to stimulate blood cell proliferation and differentiation. For example, after an ablative treatment to kill leukemic cells in the bone marrow, hematopoiesis then needs to be re-established with normal cells. Thus, GM-CSF may be part of a post-remission therapy. GM-CSF binds to GM-CSF receptors of the cells. Other cytokines, such as IL-3 have their own receptors.

For the production of diagnostics and therapeutics, the development of immune responses is used to generate both reagents and cells with potential therapeutic effects and diagnostic utility. The difficulty with this approach is that tumor cells or other abnormal cells that flourish are typically not very antigenic or easily recognized by the immune system, otherwise the tumor would not grow and flourish in a patient. For this reason, reliance on the immune system to develop molecules which specifically bind to malignant cells is highly problematic.

Thus, there is a need for materials and methods to generate potential carrier molecules that are not recognized as antigens by the immune system. There is also a need for molecules that can identify abnormalities on the surfaces of malignant cells that do not rely upon antigenicity. There is an additional need for the development of carrier molecules whose binding to tumor target cells is not dependent upon the presence of highly immunogenic molecules on the surface of the tumor cell.

Further, there are needs for: methods to bind cells in a specific manner that permits specific identification of cancerous cells (e.g. normal, AML, CML and other types of cancer cells) and/or the stage of disease (e.g. MDS, in remission, acute leukemia and other types of malignant cells); methods to study binding to cancer cells that more closely approximate cancer cells in vivo than the immortalized cancer cell cultures; cell-specific binding materials and methods that are useful for developing diagnostic tools and markers, and for developing therapies; and alternative means to specifically stimulate or inhibit cellular proliferation and/or differentiation.

### BRIEF SUMMARY OF THE INVENTION

The present invention provides materials and methods for assaying peptide binding to cells and can determine whether any binding is cell-specific. The present invention further provides materials and methods for assaying peptide binding to cancer cells, including leukemic cells and tumors of various kinds of cancer.

The present invention further provides the use of cell-binding peptides for cellular targeting of compounds, such compounds including labels, therapeutic and cytotoxic compounds. The present invention provides cell-binding peptides to up- or down-regulate cellular proliferation and/or differentiation, including affecting the biological activity of cytokines and/or their receptors.

The present invention discloses several specific cell-binding peptides that permit distinction between cancerous and non-cancerous cells, such as leukemia cells, their type and/or stage of leukemia progression.

The present invention contemplates manifold uses of peptides demonstrating specific binding properties, including their use for directing targeted drug delivery to cells and targeted detection/identification of cells. Thus, the present invention contemplates the binding of a peptide to a target cell or cellular component. The invention contemplates many embodiments of the peptide, either alone, or as part of or attached to a carrier molecule to make a specific carrier molecule. The invention also contemplates the attachment of additional moieties to the specific carrier molecule, including identification tags (e.g. fluorescent or radioactive markers), components of an identification system (e.g. horseradish peroxidase or an antibody), or cell treatments (e.g. doxirubicin or daunomycin).

This capability for targeted direction to specific cell types can also be used for protection of normal cells, such as with an antidote to a treatment for malignant target cells. The capability for targeted direction also provides a means for binary treatments where two components of a treatment, dangerous together, are kept separate until they are brought together at the target by specific direction to the target cells.

The present invention has many benefits and advantages, several of which are listed below although not every embodiment of the present invention necessarily conveys all of the enumerated benefits and advantages.

One benefit of the invention is that, peptides are disclosed that bind differentially to different types of cells.

An advantage of the invention is that peptides that bind differentially to normal cells versus leukemia cells can be used for targeted drug delivery diagnostic and therapeutic methods.

Another benefit of the invention is that peptides that bind differentially to different types of leukemia cells can be used to classify disease types.

Another advantage of the invention is that peptides that bind differentially to different cell types can be used for preparing specific separation materials and methods.

A further benefit of the invention is that cell specific binding peptides can be used to direct treatment to malignant cells, reducing negative effects to non-malignant cells, such as normal bone marrow cells and normal B cells.

A still further advantage of the invention is that it provides a means for in vivo “magic bullet”-style targeted delivery of drugs to kill malignant cells, while having a less toxic effect on non-malignant cells in a mammal.

Still further benefits and advantages of the various embodiments of the present invention will be apparent to a person of ordinary skill from the description that follows.

### BRIEF DESCRIPTION OF THE DRAWINGS

In the drawings forming a portion of this disclosure,

FIG. 1 illustrates the effects of several peptides on the differentiation of the human myeloid leukemia cell line HL60. The data are shown for DMSO alone as a control for differentiation, free peptides G5 12-8B and A2 11-24, the G5 and A2 peptides as displayed by phage, the two free G5 and A2 peptides combined, the two phage-displayed G5 and A2 peptides combined, controls of the M13 phage that was not displaying any exogenous peptide in 2 different concentrations, and the free peptides G5 and A2 at high peptide concentrations. The data show that both free peptides G5 and A2 stimulate the differentiation of human myeloid leukemia HL60 cells. The y-axis shows the percent of differentiated cells for each exemplified peptide.

FIG. 2 shows the effect of various peptides on cellular proliferation. The number of cells after 7 days culture growth were counted in the presence or absence of the different molecules. Results are shown for the free G5 12-8B peptide alone (artificially synthesized), phage displaying G5 12-8B, the free A2 11-24 peptide, phage displaying the A2 11-24 peptide, a combination of the free G5 and A2 peptides, a combination of the free pure with phage displaying the peptides, and several concentrations of peptide.

FIG. 3 shows the effect of various peptides on the viability of cells grown under various conditions for 5, 7 or 9 days, discussed in Example 12. Studies were conducted of the effects of the free G5 12-8B (artificially synthesized), phage displaying G5 12-8B, the free A2 11-24 peptide, phage displaying the A2 11-24 peptide, a combination of the free G5 and A2 peptides, a combination of pure peptides plus phage displaying the peptides, and several concentrations of peptide.

FIG. 4 shows the total binding curve (squares and dashed line) for cell binding of the peptide A2 having SEQ ID NO:3 to patient bone marrow cells as evaluated by an ELISA assay using biotin-conjugated peptide, as discussed below in Example 14. The data demonstrate that the binding intensity increased with the concentration of the peptide until a maximum binding concentration is obtained. The binding of the peptide can be competed by a 100-fold excess of non-labeled peptide (SEQ ID NO:3). The non-specific binding component (circles and solid line) is shown by competition of biotinylated peptide by binding with an excess of peptide.

FIGS. 5A-5E show fluorescence binding studies performed with AML cells confirming the binding of peptide A2 having SEQ ID NO:3, as discussed below in Example 14. The peptide was visualized by fluorescence microscopy. FIGS. 5A, 5B and 5C show the AML bone marrow cell controls with the cells alone, the cells after incubation with streptavidin-FITC and the cells after incubation of the cells with biotin and streptavidin-FITC. FIG. 5D shows AML bone marrow cells after incubation of the cells with a biotin-peptide A2 (SEQ ID NO:3) conjugate and streptavidin-FITC. Some cells were clearly not stained, indicating that one or more sub-populations of cells express a target for this peptide. FIG. 5D, in comparison with the control with biotin alone (FIG. 5C) showed that the binding is via the peptide and not the biotin conjugate. In FIG. 5E, free peptide A2 (SEQ ID NO:3) was also present. The presence of an excess of peptide decreased the intensity of the staining of the biotin-peptide suggesting specific binding to the cells. Similar results were obtained with other patient samples. A slight signal was seen in normal bone marrow that was not competed out by an excess of peptide. This suggests that that weak signal is due to a non-specific interaction between the peptide and some other cellular targets.

FIGS. 6A-6E show fluorescence binding studies performed with normal cells confirming the lack of binding of peptide A2 having SEQ ID NO:3, as discussed below in Example 14. The peptide was visualized by fluorescence microscopy. FIGS. 6A, 6B and 6C show the normal bone marrow cell controls with the cells alone, the cells after incubation with streptavidin-FITC and the cells after incubation of the cells with biotin and streptavidin-FITC. FIG. 6D shows AML bone marrow cells after incubation of the cells with a biotin-peptide A2 (SEQ ID NO:3) conjugate and streptavidin-FITC. The cells were clearly not stained, indicating that normal cells do not express a target for this peptide. In the sample of FIG. 6E, free peptide A2 (SEQ ID NO:3) was also present. The presence of an excess of peptide did not noticeably affect the already low intensity of the staining of the biotin-peptide suggesting no specific binding to the cells. The lack of binding to normal cells coupled with the results of FIG. 5 shows that peptide A2 binds specifically to leukemia cells rather than normal cells.

FIG. 7 shows percentages of the various types of cells in a morphology evaluation after a 14-day incubation of AML isolated cells in methylcellulose culture with the listed combinations of peptides, as discussed below in Example 15. The solid black bars show the percentage of immature cells in the culture. The solid white bars show the percentage of small myeloblasts in the culture. The dark gray bars show the percentage of young cells in the culture. The light gray bars show the relative percentage of monophages to macrophages in the sample.

FIG. 8 shows the percentages of the various types of cells from a morphology evaluation of the cells after 14 days of culture in a methylcellulose medium of AML bone marrow cells while being exposed to peptide A2. The solid black bars show the percentage of immature cells in the culture. The solid white bars show the percentage of small myeloblasts in the culture. The dark gray bars show the percentage of young cells in the culture. The light gray data bar shows the relative percentage of monophages to macrophages in the sample. The data are discussed below in Example 15.

FIG. 9 shows the percentages of the various types of cells from a morphology evaluation of the cells after 14 days of culture in a methylcellulose medium of AML bone marrow cells while being exposed to peptide A2. The solid black bars show the percentage of immature cells in the culture. The solid white bars show the percentage of small myeloblasts in the culture. The dark gray show the percentage of young cells in the culture. The light gray data bar shows the relative percentage of monophages to macrophages in the sample. The data are discussed below in Example 15.

FIG. 10 shows the effect of the peptides on cellular proliferation. The number of colonies growing in methylcellulose culture after 14 days exposure to the listed peptides is shown for every 10,000 cells initially inoculated for normal bone marrow cells. The data are discussed below in Example 15.

FIG. 11 shows the effect of the various peptides on differentiation of the cells. The percentages of the various types of cells from a morphology evaluation of the cells after 14 days of culture in a methylcellulose medium of normal bone marrow cells while being exposed to the listed peptides. The solid black bars show the percentage of immature cells in the culture. The solid white show the percentage of young cells in the culture. The light gray data bar shows the relative percentage of monophages to macrophages in the sample. The right diagonally-hatched bars show the percentage of granulocytic cells. The left diagonally-hatched bars show the percentage of lymphocytes in the culture. The data are discussed below in Example 15.

### DETAILED DESCRIPTION OF THE INVENTION

The present invention provides materials and methods for assaying the binding of peptides to cells. The invention also provides for the determination of the cell binding specificity of peptides. The invention further provides for the analysis of the biological effects of peptide binding to cells. The invention contemplates the exploitation of peptides that bind to cells, particularly for diagnostic and therapeutic purposes.

Discussed below are several peptides that have been identified as having specific cell binding activity. Several uses of the peptides are also discussed in detail, including uses in diagnostics and kits and therapeutic uses.

Peptides that bind differentially to cells, such as those disclosed herein or those identified using the methods disclosed herein, are useful in a wide variety of applications. By differential binding to cells is meant that the peptides bind preferentially or specifically to some cells and not to others. Examples of several such peptides are disclosed below. Methods of obtaining such peptides are disclosed herein. A useful library of peptides to screen for such peptides is also disclosed herein.

I. Cell-Binding Peptides

The present invention contemplates various uses of peptides that bind differentially to cells, specifically to some cells and not to others. A number of peptides are disclosed herein with a range of cellular binding specificities.

The present invention provides isolated peptides that have differing binding characteristics for a variety of hemopoietic cells including normal bone marrow cells, acute and chronic leukemia cells, preleukemia cells, cord blood cells, cord blood stem cells, chronic lymphocytic leukemia cells, subsets of lymphoid cells, and normal peripheral blood cells.

Disclosed herein are the amino acid sequences of several peptides that were found to bind to different kinds of cells. The cell-specific binding can be exploited in a wide variety of applications.

| Amino Acid Sequence | SEQ ID NO: | Number of Peptide |
|---|---|---|
| EFQQWSGK | SEQ ID NO:1 | G5 12-8B no Cys |
| CEFQQWSGKC | SEQ ID NO:9 | G5 12-8B |
| NHVCSRLG | SEQ ID NO:2 | B6 12-8B no Cys |
| CNHVCSRLGC | SEQ ID NO:10 | B6 12-8B |
| IEETARKG | SEQ ID NO:3 | A2 11-24 no Cys |
| CIEETARKGC | SEQ ID NO:7 | A2 11-24 |
| CIEETAAKGC | SEQ ID NO:8 | A2A |
| NNATVEDE | SEQ ID NO:4 | C8 12-8B no Cys |
| CNNATVEDEC | SEQ ID NO:13 | C8 12-8B |
| HSWKPDKL | SEQ ID NO:5 | D2 11-24 no Cys |
| CHSWKPDKLC | SEQ ID NO:17 | D2 11-24 |
| ETGERIVL | SEQ ID NO:6 | D4 12-8A no Cys |
| CETGERIVLC | SEQ ID NO:18 | D4 12-8A |
| NNATFEDG | SEQ ID NO:21 | G2 12-8A no Cys |
| CNNATFEDGC | SEQ ID NO:12 | G2 12-8A |
| NELHMKQH | SEQ ID NO:15 | G7 12-8B no Cys |
| CNELHMKQHC | SEQ ID NO:11 | G7 12-8B |
| DEKRGPNEC | SEQ ID NO:16 | B4 C10 no Cys |
| CDEKRGPNEC | SEQ ID NO:14 | B4 C10 |
| NETTVREY | SEQ ID NO:19 | A6 11-24 no Cys |
| CNETTVREYC | SEQ ID NO:20 | A6 11-24 |
| VSEDIYDA | SEQ ID NO:22 | E2 12-8A no Cys |
| CVSEDIYDAC | SEQ ID NO:23 | E2 12-8A |
| CGREGEDW | SEQ ID NO:27 | G2 9-10-01 no Cys |
| CCGREGEDWC | SEQ ID NO:26 | G2 9-10-01 |
| KRGIHPES | SEQ ID NO:29 | B3 9-10-01 no Cys |
| CKRGIHPESC | SEQ ID NO:28 | B3 9-10-01 |
| KRGIHPES | SEQ ID NO:29 | F4 9-10-01 no Cys |
| CKRGIHPESC | SEQ ID NO:28 | F4 9-10-01 |
| QPTQYVMK | SEQ ID NO:30 | F4 9-10-01 no Cys |
| CQPTQYVMKC | SEQ ID NO:31 | F4 9-10-01 |

Polypeptide molecules are said to have an “amino terminus” (N-terminus) and a “carboxy terminus” (C-terminus) because peptide linkages occur between the backbone carboxy group of a first amino acid residue and the backbone amino group of a second amino acid residue. Typically, the terminus of a polypeptide at which a new linkage would be to the carboxy-terminus of the growing polypeptide chain, and polypeptide sequences are written from left to right beginning at the amino terminus. As used herein, “near” the N- or C-terminus refers to within about 10 amino acid residues of the terminus.

All amino acid residues identified herein are in the natural L-configuration. In keeping with standard polypeptide nomenclature, abbreviations for amino acid residues are in agreement with common usage and World Intellectual Property Organization (WIPO) Handbook on Industrial Property Information and Documentation, Standard ST.25: Standard for the Presentation of Nucleotide and Amino Acid Sequence Listings in Patent Applications (1998) including Tables 1 through 6 in Appendix 2, herein incorporated by reference.

Also contemplated are substitutions of one amino acid for another. Conservative substitutions are those in which one amino acid residue is replaced by another, biologically similar residue. Conservatively substituted versions of the above-listed peptides constitute a preferred alternative embodiment of the present invention. Examples of conservative substitutions include the substitution of one hydrophobic residue such as isoleucine, valine, leucine or methionine for one another or one of phenylalanine or tryptophan for one another, or the substitution of one polar residue for another as between arginine and lysine, between glutamic and aspartic acids or between glutamine and asparagine.

Alternatively, a variant includes “nonconservative” changes, e.g., replacement of a glycine with a tryptophan. Analogous minor variations can also include amino acid deletions or insertions, or both. Guidance in determining which amino acid residues can be substituted, inserted, or deleted without abolishing biological activity can be found using computer programs well known in the art, for example LASERGENE software (DNASTAR Inc., Madison, Wis.).

As used herein, a “specific carrier molecule” is a molecule that can specifically bind to some cells and not to other cells. The cells to which a specific carrier molecule binds is a “target cell”. A target cell population may be intermixed with other cells.

Accessory molecules are attached to specific carrier molecules to make it possible to deliver specific molecules to a target cell population while non-target cells in the immediate environment of the cell targets will not bind the “accessory” molecules.

A variety of accessory molecules with different characteristics can be attached to the carrier molecule and can confer upon the carrier molecule the potential for making specific identification of a few cells out of a plurality of other cells. A variety of effector molecules can also be carried to the target cells with potential of specifically altering the behavior or state of the target cells. Additionally, it is possible that the simple binding to the cell target of the specific carrier molecule may alter the behavior of the target cells. Contemplated applications of cell-specific carrier molecules are discussed in further detail below.

II. Cell-Binding Peptide Diagnostics

Patient diagnosis. The present invention contemplates the use of the cellular specificity of the binding of peptides to different leukemia cell populations for a reliable classification of patients. This embodiment is useful for tailoring a treatment regime which is specific to patient or disease.

Measurement of the relative amounts of different leukemia cell populations and preleukemia cell populations are useful in assaying the patient's prognosis and deciding on a course of treatment, as well as for monitoring the patient's response to treatment.

Patient monitoring. The invention further contemplates the use of peptides as diagnostic tools capable of detecting and quantifying residual malignant cells in patients both during and after treatment.

Patient prognosis. The invention also contemplates the ability to prognosticate the likely behavior of preleukemia or leukemia in patients using a hierarchy of peptide binding of marrow cells which have been associated with the future behavior of the illness. Myelodysplastic cells and myeloproliferative cells are types of preleukemic cells.

Cell separation and quantification. The invention contemplates tools for separating specific cell types from mixed cell populations (leukemic or normal) using solid state methods (columns or panning), flow cytometry or magnetic beads. Thus, the present methods provide for attaching a peptide to a solid support. The solid support thus derivatized with a cell-specific binding capacity could be used to separate samples of cells. For example, exposing a mixed population of cells to a solid support derivatized with a disclosed peptide (such as passing a liquid solution through a column, or slurrying in a batch treatment, panning or exposing derivatized sample wells) is useful for obtaining a target cell population that is free of contamination by other cells.

1. Use of the specific carrier molecule to identify and facilitate recovery of the target cells-diagnostic uses and cell recovery uses

The attachment of a “tag” to a peptide that specifically binds to a cell population makes it possible to determine whether a cell population is present, measurement of the cell population. This embodiment can be useful to measure the presence of residual disease after treatment or the reappearance of malignant cells after they have been unrecognizable for a period of time. Tag molecules include both fluorescent and non-fluorescent markers such as peroxidase, alkaline phosphatase and other similar markers. These markers can then be used to both stain biopsies and disaggregated cell populations using light and electron microscopy. The attachment of suitable dyes to the peptide makes it possible to study cells by flow cytometry and separate and recover the labeled target cells by cell sorting.

Flow cytometric analysis permits cell visualization on equipment that is widely commercially available. For example, the FACSort flow cytometer (Becton-Dickinson Immunocytometry Systems, San Jose, Calif.) is typically interfaced to a PC, such as the Consort 32 workstation (Hewlett-Packard). In an example using fluorescence to visualize cells, a single 15 mW argon ion laser beam (488 nm) is used to excite the FITC fluorescence, and fluorescence data is collected using 530 nm band pass filter to provide a histogram. The percent of fluorescent cells can be calculated, for example using Lysis II software.

The methodologies described above can be used for diagnostic purposes, to monitor the effects of therapy and to identify residual disease or regrowing disease in the bone marrow or peripheral blood or body fluids obtained from patients (including the blood and the CSF). They are also useful for prognostic purposes. For example, the presence or absence of a receptor on a cell surface which can distinguish between cells associated with different prognoses would be possible. The appearance of binding to peptides which are associated with lower or higher level of abnormality could be used for prognostic purposes.

The specific carrying molecule can also be attached to a solid phase (columns, the flat lower surface of a dish or plate) and used to separate and recover the target subpopulation by adherence of the target cells to the solid surface with subsequent elution of the cells from the solid phase.

III. Cell-Binding Peptide Therapies

When the peptides bind to molecules (e.g. receptors) on the surface of, or an intracellular target in, a cell they can induce the differentiation of cells, affect cell proliferation and induce cell apoptosis, either by affecting cytokines or acting alone. Such effects were observed for several of the peptides disclosed herein.

Stimulation of Differentiation. The present invention provides avenues for novel approaches to the treatment of acute myelogenous leukemia (AML) by inducing the leukemia cells to differentiate into mature cells that carry out normal cell functions, in essence converting them to a nonmalignant state. Most, if not all, AML cell populations contain a sufficient amount of genetic information to enable them to differentiate and become either nonmalignant or to have their malignant behavior significantly reduced. To date, there has been no proven method of inducing AML cell differentiation to a nonmalignant state.

The present inventors noted that the administration of chemotherapy induces the differentiation of some leukemia cells in vivo in patients, and that leukemias in which differentiation was induced during chemotherapy treatment had much longer remissions than those leukemias in which differentiation was not induced.

It has been found that a synthetic vitamin A derivative (ATRA) could induce differentiation in acute promyelocytic leukemia (a fairly uncommon leukemia). The induction of differentiation is of significant benefit to the patient and can be curative if the patient is treated with both ATRA and chemotherapy.

In some embodiments of the present invention, peptides and specific carrier molecules using those peptides are capable of inducing the differentiation of both HL60 cells and freshly-obtained human AML cells. In a preferred embodiment, bacteriophages carrying specific peptides described herein induce the differentiation of both the AML cell line HL60 and of freshly obtained human AML cells.

Modifying Proliferation. One problem that occurs when regulating the proliferative behavior of a specific cell population is that the delivery of the regulatory molecule results in the stimulation or inhibition of proliferation by all of the cell populations that are present (in the marrow, for example). This general effect often defeats the goal of the manipulation since the latter is being undertaken to make the tumor cells more sensitive to treatment either by increasing their sensitivity to the anti-tumor agent (by increasing proliferation) or by preventing their regrowth after chemotherapy has ended. The same approach can be used to alter the behavior of the residual normal marrow or GI lining cells by stimulating their proliferation between courses of treatment and inhibiting their proliferation during the administration of chemotherapy.

In some embodiments of the present invention peptides and specific carrier molecules using those peptides are capable of inhibiting the proliferation of leukemia cells. In a preferred embodiment, specific peptides described herein can severely retard the proliferation of leukemia cells. The invention contemplates obtaining the peptides through chemical synthesis or biochemical methods, such as growing the appropriate phage. In contemplated clinical applications, the chemical synthesis of the disclosed peptides is preferred through means well known in the art.

One embodiment of the present methods can be accomplished by targeting the stimulatory and inhibitory molecules (GM-CSF, IL-3, SCF, as examples of the former and H. Iran and TGF-β as examples of the latter) to normal cells to slow or prevent proliferation during chemotherapy and to stimulate proliferation between courses of therapy.

Cell-targeting agents. The invention provides methods that use peptides as specific carriers for chemotherapeutic agents, radiopharmaceuticals, and toxins which will deliver these therapeutic agents to leukemia cells and not to normal cells. These “guided therapeutic missiles” will find their use in the treatment of patients and in the removal of contaminating leukemia cells from stem cell preparations which are to be used in transplantation.

Cytotoxic Therapy. The binding of toxic agents (natural or synthetic) to specific carrier molecules permits the combination to deliver specific toxic agents directly to the targeted cell population while minimizing the exposure of non-target cell populations to the toxic agent. The approach can be used in vivo (e.g. administration to patients) as well as in vitro (e.g. for the “purging” of the cell population of targeted cells.

Cytotoxic agents which could be bound to the specific carrier molecules include radioactive substances, cell poisons which cannot be administered to patients with the expectation of therapeutic benefit, and chemotherapeutic agents. It is also possible to deliver “binary” therapy with a toxic agent attached to a carrier molecule which is targeted to the targeted malignant cell population and the “antidote” to a peptide which binds other important normal cells so that the latter are rescued from the effects of the toxic agent. An illustrative example of a binary therapy is the administration of methotrexate to the targeted malignant cells and folic acid to the normal marrow or gastrointestinal cells to optimize the therapeutic index of the antimetabolite.

Binary Therapy. The present invention provides for the exploitation of peptides that bind differentially to different cell types in the formulation of binary therapeutic strategies. Another example of a binary strategy is the use of agents that are inactive alone, but that become active when combined. Such a strategy is possible using targeted delivery, such as is possible with cell-specific binding peptides, to ferry the separate components to the site of treatment within the body, such as a malignant cell.

The present invention also provides for the use of a first peptide that binds to both leukemic and normal cells in conjunction with a second peptide that carries an antidote to the first peptide and which binds only normal cells. An example of such a binary treatment includes, but is not limited to, methotrexate and citrovorum factor.

The present invention further discloses the use of a first peptide that specifically binds to normal cells, but not to malignant cells, to stimulate the growth of normal progenitors without stimulating the proliferation of leukemia cells (or other malignant cells). The stimulation of proliferation of normal cells is also helpful for accelerating recovery from drug-induced toxicity (e.g. chemotherapy) or is useful prior to the administration of chemotherapy to raise the levels of normal cells to a point that even with a reduction in number caused by therapy a drop to dangerously low normal cell levels is prevented.

Immune System Stimulation. Specific carrier molecules are useful in methods of stimulating or inhibiting the activity of specific subpopulations of cells in the immune system. The binding of molecules to a cell can alter the behavior of the target cells, from a functional perspective and might also make the cells more immunogenic than the same cells without the bound molecule.

For example, the invention contemplates a method of enhancing the immunogenicity of a target cell using a peptide that specifically binds that target cell, e.g. a chronic leukemia cell, linked to a carrier molecule that is itself antigenic or is attached to an antigen. An illustration of this method includes binding growth or function stimulating or inhibiting cells to an appropriate carrier molecule and delivering the active molecule to a specific targeted cell population (e.g. IL2, inter alia).

One problem encountered in the art in attempts to stimulate a patient's immune system against a tumor is that while tumor-specific antigens in all likelihood do exist, the tumor cells are often only weakly- or non-immunogenic. This may be the case because only a small number of tumor-specific antigens are present on the surface of the cells or may be due to the presence of molecules on the surface of the tumor cell that block access to tumor-specific or associated antigens. In these situations, even if tumor-specific or tumor-associated antigens are present, the antigen-processing cells are not capable of taking up the tumor cells and initiating a response to these antigens.

Thus in one embodiment of the invention, antibodies directed to the specific immune cells or FC′ receptors are attached to the targeted carrier molecule to deliver the tumor cells to the immune cells. This mode of delivery facilitates the development of an immune response since the carrier molecule and the tumor cells are taken up by the immune cells with concomitant degradation of the complex and sensitization of the immune cells to tumor-associated or tumor-specific antigens to which the immune cell would otherwise not have been exposed.

The carrier molecule can be attached to an antibody directed against a killer cell or to molecules which will attract and thus bring large numbers of killers cells to the targeted cells, thus facilitating immune activity against the targeted cell population.

This same approach could be used in the treatment of autoimmune disease or graft vs. host disease by targeting the effector cells in these situations and delivering specific inhibitory or killing molecules to these cells thereby suppressing the autoimmune response.

Pharmacological considerations. The dosage regimen for treating a disease condition with a compound and/or composition useful in this invention is selected in accordance with a variety of factors, including the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed can vary widely and therefore can deviate from the preferred dosage regimen set forth above.

A mammal in need of treatment for leukemia can receive one or more administrations of a specific carrier molecule carrying the selected treatment molecule until the desired clinical outcome is achieved. Thus, for example, multiple doses of a specific carrier molecule can be administered until there is a reduction in symptomology, or until diagnostic analyses demonstrate that the leukemia is in remission or cured. Cytokine or growth factor derivatized specific carrier molecule administration can also utilize multiple administrations until the desired clinical outcome is achieved. For example, growth factor or cytokine-linked specific carrier molecule administration can continue until flow cytometry indicates an effect on the amount of targeted cells.

The contacting of a peptide or treatment-derivatized specific carrier molecule with a target cell population, such as a neoplasm of B cell lineage can be performed ex vivo or in vivo. Ex vivo contacting of a peptide or specific carrier molecule with a B cell neoplasm can be done by exposing neoplastic B cells to an effective number (amount) of a peptide or treatment-derivatized specific carrier molecule, as described in the Examples elsewhere herein. The amounts of the treatment without the cell-specific guidance system of the invention are well known in the art. It is expected that in using a method of the present invention, lower dosages would be necessary, but can be worked out routinely by a worker of ordinary skill in the art.

In vivo contacting of a peptide or treatment-derivatized specific carrier molecule with a target cell population can be done by administering an effective amount of a peptide or treatment-derivatized specific carrier molecule to a human, preferably parenterally, by methods that are well known in the art. For example, administration of a peptide or treatment-derivatized specific carrier molecule is preferably performed by subcutaneous administration. However, it is also contemplated that the peptide or treatment-derivatized specific carrier molecule can be administered by continuous intravenous infusion, for example, via an implantable or external continuous infusion pump fitted with an appropriate catheter.

The treatment of a target cell population is preferably accomplished in a human by a method of the invention. A treatment method contemplated by the invention is used for treating a host mammal such as a mouse, rat, rabbit, dog, horse, primate such as a monkey, chimpanzee or human that has a condition requiring the treatment method.

A compound useful in the present invention can be formulated as a pharmaceutical composition. Such a composition can then be administered parenterally in dosage unit formulations containing conventional nontoxic pharmaceutically acceptable carriers and vehicles as desired. The term parenteral as used herein includes subcutaneous administration, intravenous administration, or local infusion techniques. Formulation of drugs is discussed in, for example, Hoover, John E. (ed.), Remington's Pharmaceutical Sciences (18th Edition), Mack Publishing Co., Easton, Pa., 1990 and Liberman, H. A. and Lachman, L., Eds., Pharmaceutical Dosage Forms, Marcel Decker, New York, N.Y., 1980. As discussed elsewhere herein, the administration of a peptide or treatment-derivatized specific carrier molecule of the invention is typically by subcutaneous or intravenous administration.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration, as is well known in the art. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

A compound useful in the present invention can also be formulated into liposomes, as discussed in Hoover, John E. (ed.), Remington's Pharmaceutical Sciences (18th Edition), Mack Publishing Co., Easton, Pa., 1990, p. 1691. Liposomes are formed by dispersing phospholipids in an aqueous medium. Water- or lipid-soluble substances such as a peptide or treatment-derivatized specific carrier molecule of the invention can be entrapped in the aqueous space within a liposome, or within the lipid bilayers of the liposome, respectively. Thus, a growth factor can be formulated into liposomes for use in a method of the invention using techniques that are well known in the art.

As noted above, the amount of active ingredient that can be combined with the carrier materials to produce a single dosage form varies depending upon the host treated and the particular mode of administration. The peptide can be formulated so that it is present on one surface of the liposome conferring binding specificity on the liposome.

IV. Cell-Binding Peptide Identification

Identification of Receptors. The invention contemplates the ability to obtain the specific cytoplasmic or membrane receptor with which a peptide interacts by fractionating cells, for example using a solid and running the fragments through a column or onto a solid structure such as a plate to which the specific peptide has been bound. The acquisition of these receptors will permit detailed study of the structure and function of the receptors and will also permit the receptors to function as immunogens under highly favorable conditions in vitro (for example, the direct immunization of dendritic cells).

Not wishing to be bound by theory, a hypothesis was considered that a large number of functionally important molecules exist on the surface of leukemia cells (and on other normal and malignant cells as well) that have not yet been identified. Possibly many of these functionally important molecules are receptors or parts of receptors that, when bound by a ligand, alter the behavior of the leukemia cells. Some hypothesize that perhaps one of every one million peptides will bind to cells and alter their behavior.

The present invention applies some recently developed technologies to obtain peptide libraries of extremely high diversity. An example of one embodiment of the present invention made thus far has about 30 million different unique peptides in the library.

The present invention contemplates methods and materials to recover bacteriophages carrying specific peptides with specific cell binding characteristics.

The present invention contemplates methods and materials conferring the ability to rapidly and simultaneously test the binding specificity of a large number of peptides against panels of target cells.

The present invention provides materials and methods for the identification of cell-surfaces that do not rely on antigenicity. The present invention utilizes a random peptide library to identify molecules which specifically react with molecules or groups of molecules on the surface of tumor cells. In this approach, a random library consisting of an extremely large number of peptides is exposed to the target cell and those peptides that selectively bind to the target cell and not other cells are recovered.

Several peptides were identified using these methods that are specific for various blood cells. Those peptides are optionally attached to carrier molecules whose binding to tumor target cells are not dependent upon the presence of highly immunogenic molecules on the surface of the tumor cell. As used herein, an immunogenic molecule refers to a molecule that will provoke a measurable immune response under normal (e.g., in the absence of disease) conditions in a subject. A non-immunogenic molecule refers to a molecule that provokes no immune response under normal conditions. A weakly immunogenic molecule refers to a molecule that fails to induce immunity against that molecule, particularly antileukemic immunity.

The following examples are offered to further illustrate, but not limit the present invention.

### EXAMPLE 1

Screening of a Peptide Library for Leukemia Cell Specificity

Binding of cells to phage displaying a peptide library was assayed to investigate interactions between the various peptides and cell surfaces to identify whether any peptides demonstrate specificity for leukemia cells, or a specific type of leukemia cell.

A peptide display phage library was screened for binding to different types of cells. Phage display technology to display peptides on the outside of a phage particle is disclosed in Jeffrey W. Smith and Erkki Ruoslahti, in “Harvesting Molecular Diversity-Biology's New Commodity”, Biotechnol. and Genet. Eng. Rev., 14:51-65 (April 1997); Diane J. Rodi and Lee Makowski, in “Phage-display technology—finding a needle in a vast molecular haystack,” Current Opinion in Biotechnology, 10(1):87-93 (1999) (construction of phage display peptide libraries using M13).

Preliminary experiments were performed to identify specific peptides by using cells from patients diagnosed with CLL, CML, and AML. The screening was performed by incubating the cells with the phage-peptides from the library. This incubation allows interaction between the peptide and the cell. The binding step was followed by several washes to eliminate free phage. Phage bound on the cell surface was eluted. The phage in the eluate was amplified to increase the phage number, thus enriching the displayed peptide pool with peptide that had bound to that cell type. The amplified eluate obtained was used for another round of binding with the same cell type to further enrich the displayed peptide pool with peptides that bind to those cells.

After the last round of binding with one cell type, the eluted phage was plated on an agar plate with the appropriate selection antibiotics. Each individual colony, corresponding to one individual peptide sequence, was amplified in a 96-well plate. After purification, these peptides were tested for their ability to bind to the cells in a microtiter plate by ELISA.

Each peptide was dispensed in a 96-well plate, containing cells previously fixed. Each colony was tested in a triplicate assay with the appropriate controls. After incubation of cells with an individual clone, the wells were washed to eliminate free phage. The bound phages were detected by using a monoclonal anti-M13 antibody/horseradish peroxidase (HRP) conjugate. After addition of the HRP substrate, the intensity of the coloration was measured by spectrophotometry. The light absorbance values (optical density values, O.D. values) are proportional to the number of phage bound to the cells.

The binding to various cell types were examined to evaluate the cellular specificity of the phage clones. For example, the clones from phage eluted from CLL patient cells were assayed for their ability to bind cells from an AML patient. Cells from normal donors (non-leukemic) were also evaluated to ascertain leukemia cell specificity.

The sequence of the peptide expressed by the phage was determined by sequencing the phage DNA. Potentially useful peptides are synthesized and examined further for specificity and biological activity.

### EXAMPLE 2

Fixation of Cells on 96-well Plates Using Poly-L-Lysine

Peripheral blood and bone marrow samples were collected from patients after informed consent. The samples were collected in tubes containing sodium citrate. Mononuclear cells were isolated by Ficoll-Hypaque density gradient separation and collection of the cells at a density of 1.077 g/mL.

The wells of a 96-well plate were pre-treated with 100 μL of poly-L-lysine (0.01 mg/mL in phosphate-buffered saline, 120 mM NaCl, 2.7 mM KCl, 10 mM phosphate buffer, “PBS”) for 2-16 hours at room temperature. After draining the poly-L-lysine from the wells, the excess poly-L-lysine was removed from the wells by flicking. The cells were washed with PBS, then suspended in PBS and diluted to a final concentration of 1.25 cells/mL. The cell suspension (200 μL; 250,000 cells) were added to each well needed for the assay.

The plates were centrifuged at 500 g for about 5 minutes in a microplate carrying centrifuge when available. The cells were fixed to the wells through the addition of glutaraldehyde (100 μL of 0.5%) and incubating at room temperature for about an hour. The glutaraldehyde was removed and the cell-coated wells washed once with PBS. Alternatively, the cells were permitted to settle to the well bottoms for about 4 hours at 37° C., and the glutaraldehyde fixative was added after removing the PBS.

A glycine solution (200 μl of 0.1 M glycine in PBS) containing bovine serum albumin (BSA; 1%) was added to the wells and permitted to incubate for 30 minutes to block excess glutaraldehyde. The glycine was removed, and the cell-coated wells washed once with PBS. A sodium pyrophosphate solution (200 μL of 0.3 mg/mL NaPPi) was added to the wells and incubated for an hour at room temperature to neutralize lysine charges. The pyrophosphate solution was removed and the wells washed once with PBS. Multiwell microtiter plates with cell-coated wells were stored at 4° C. with fresh PBS solution in the coated wells.

### EXAMPLE 3

Preparation of Peptide Libraries

A library of E. coli infected with M13 phage having p3+3 mutants having leukemia protein peptide insertion fragments (“pSKAN libraries in E. coli”) (Display Systems Biotech, Denmark) was stored frozen. The E. coli were inoculated (200 μL) into Luria-Bertani medium (LB medium, 20 mL in a 125 mL flask) under ampicillin (20 μL of a 250 mg/mL solution) and tetracycline (20 μL of a 20 mg/mL solution), and incubated overnight (8 to 16 hours) at 37° C. with agitation. The overnight cultures (1 mL) were then used to inoculate larger cultures of LB medium (100 mL in a 1 L flask) under ampicillin (100 μL of 250 mg/mL solution) and tetracycline (100 μL of 20 mg/mL solution). The remainder of the overnight cultures was transferred into Eppendorf® tubes in aliquots with an equal volume of glycerol and stored at −80° C. or −140° C. (phagemids).

The cultures were incubated at 37° C. with agitation until the optical density at 600 nm of a one centimeter path length of the culture was 0.5 absorbance units (about 3 hours), then a mixture of helper phages (500 μL of a 10¹⁰ to 10¹¹ cfu/mL solution) was added to the culture. The cultures where incubated at 37° C. with helper phage for 15 minutes without agitation, then with agitation for an hour. At the end of the hour, kanamycin (100 μL of a 50 mg/mL solution) was added, and the cultures incubated with agitation overnight (8 to 16 hours).

The following morning, the cells were separated by centrifuging the cultures for 10 minutes at 8,000 rpm. The supernatant was decanted into a fresh tube and centrifuged for another 10 minutes at 8,000 rpm to eliminate all bacteria. A polyethylene glycol/sodium chloride solution (4 mL PEG/NaCl for every 20 mL of supernatant) was added to precipitate the phage from the supernatant for at least two hours on ice. The PEG/NaCl-treated supernatant was centrifuged (30 minutes at 10,000 rpm), the supernatant was decanted and the precipitate was recentrifuged and supernatant removed again to more completely remove the supernatant.

The phage precipitate pellet was dissolved in 1 mL of PBS buffer and the resulting phage solution transferred to an Eppendorf® tube and centrifuged for 10 minutes at 13,000 rpm. The solution was pipetted off the undissolved precipitate, aliquoted into Eppendorf® tubes and titered.

### EXAMPLE 4

Binding of Phage and Cell

The cells were washed one time with an incubation buffer comprising of a PBS and Ca [1 mM] solution and containing Mg [10 mM]/Milk (0.1%). The cells were counted and evaluated for viability by the trypan blue dye exclusion test. The cells were diluted 1:2 in trypan blue (4%) and counted on hemocytometer after 5 minutes. The dead cells retained the blue dye.

The cells were then suspended in the incubation buffer at the appropriate concentration and then phage was added to the suspended cells. The cell suspension with the phage was incubated for about 1.5 hours at 4° C.

After incubation, the cells were washed 5 times with the incubation buffer. Between the third and fourth wash and between the fourth and fifth wash the tube was changed to eliminate the phage binding to plastic.

Acid elution buffer (100 μL) was added to the cell suspension with the phage, and then incubated for 10 minutes on ice. The cell suspension with phage was then centrifuged. After centrifugation, the supernatant was transferred into an Eppendorf® tube containing tris 1 M pH=8 (20 μL) for the purpose of neutralization. This elution step was repeated and the supernatants were pooled together. Buffer (Tris/EDTA; 1 mL) was added to the pellet to lyse the cells. The lysate was retained for amplification.

### EXAMPLE 5

Phage Titering for Display

An E. coli (WK61mutS) culture was prepared by placing Luria-Bertani medium (“LB”; 5 mL) under tetracyline (20 μg/ml) and incubated overnight (8 to 16 hours).

The overnight E. coli culture (100 μL) was added to the LB medium (10 mL) containing tetracycline (20 μg/mL), and incubated until a light absorbance value (optical density value, O.D. value) at 600 nm of 0.5 absorbance units by spectrophotometry was measured. This E. coli culture (100 μL) was added to diluted phage (100 μL), and was incubated at 37° C. for 30 minutes.

The phage-infected bacterial culture (200 μL) was dispensed in three petri dishes. The first dish (“LB/Amp/Tc”) contained LB medium with ampicillin (250 μg/mL) and tetracycline (20 μg/mL), the second dish (“LB/Kan/Tc”) contained LB medium, kanamycin (50 μg/mL) and tetracycline (20 μg/mL) and the third dish (“LB/Tc”) contained LB medium and tetracycline (20 μg/mL). The petri dishes were incubated at 37° C. overnight and the number of colonies was counted.

### EXAMPLE 6

Amplification of Eluate

An overnight culture of the phage-infected E. coli (WK61mutS) culture from Example 5 was used to inoculate LB medium (5 mL). The overnight culture was incubated overnight (8 to 16 hours) under tetracyline (20 μg/ml) selection.

A. Reinfection of Bacteria

The overnight E. coli culture (100 μL) was used to inoculate 10 mL of LB medium containing tetracycline (20 μg/mL), and incubated with shaking at at 37° C. until an O.D. value of 0.5 was reached (about mid-log phase). This culture was added to the amplified eluate and incubated for 30 minutes at 37° C. without shaking to permit cell re-infection by the phage eluted from the cell-binding assay.

After the bacterial culture had been re-infected, the phage-reinfected bacteria were collected by centrifugation (Beckman® tube) for 5 minutes at 8,000 rpm. The phage-reinfected cell pellet was resuspended in LB medium (400 μL) containing ampicillin (250 μg/mL) and tetracycline (20 μg/mL). The culture is then dispensed in two petri dishes, containing LB/amp/Tc and incubated overnight at 37° C.

B. Packing

The reinfected bacteria was suspended into LB/Amp/Tc (20 mL). The reinfected bacteria suspension (2 mL) was used to inoculate LB/Amp/Tc (50 mL in a 250 mL flask) and was incubated with shaking at 37° C. for an hour. Aliquots of reinfected bacteria were prepared for storage by layering with glycerol (500 μL of suspension +500 μL of glycerol).

Helper phage stock solution (100 μL) (10¹¹-10¹² cfu/mL) was added to suspension and incubated for 15 minutes at 37° C. without shaking. The suspension was incubated 1 hour with shaking before addition of kanamycin (50 μg/mL) with further incubation overnight.

C. Phage Purification

The overnight culture was centrifuged at 8,000 rpm for 10 minutes. The phage-containing supernatant was retained and centrifuged again to clarify. The phage-containing supernatant was mixed with PEG/NaCl (1 volume unit for every 5 volume units) and incubated on ice for at least 2 hours. After incubation, the supernatant centrifuged again at 10,000 rpm for 30 minutes and the phage pellet was resuspended in PBS (1 mL). The resuspended pellet was transferred to an Eppendorf® tube and centrifuged for 10 minutes at 13,000 rpm. The supernatant was maintained at 4° C. Aliquots were diluted with an equal volume of glycerol for storage at −140° C.

### EXAMPLE 7

Amplification of Individual Phage

A. Amplification of Colonies Stored at 4° C.

LB medium (2 mL) containing tetracycline (20 μg/mL) and ampicillin (250 μg/mL) “LB/Amp/TC” was inoculated with one colony of E. coli containing pSKAN phagemid. The starter culture was incubated at 37° C. with agitation until the culture reached saturation or overnight. The saturated, phage-infected culture (20 μL) was inoculated into LB/Amp/TC medium containing helper phage M13KO7 (0.2 μL of a 10¹¹-10¹² cfu/mL stock solution) and warmed at 37° C. without agitation for 15 minutes to permit infection by the helper phage. After infection, the reinfected bacterial culture was incubated for 1 hour with agitation. After the hour incubation, kanamycin (50 μg/mL) was added. The culture should have been only slightly turbid, so if growth was too vigorous, the culture was diluted with prewarmed LB medium until the turbidity was only slightly visible.

The culture was again incubated at 37° C. with agitation overnight and phage were purified as previously described (Example 6C).

B. Amplification of Individual Colonies

LB medium (2 mL) containing tetracycline (20 μg/mL) and ampicillin (250 μg/mL) “LB/Amp/TC” was inoculated with one colony of E. coli containing pSKAN phagemid. Helper phage M13KO7 (2×107 cfu/mL concentration/0.2 μL of a stock solution at 10¹¹-10¹² cfu/mL) was added and the cells incubated at 37° C. with agitation for 1-1.5 hours. The culture should have been slightly turbid, and if growth was too vigorous the culture was diluted with pre-warmed LB medium until the turbidity was only slightly visible.

Kanamycin (50 μg/mL) was added to the culture and it was incubated overnight at 37° C. with agitation, and phage was purified as previously described (Example 6C).

### EXAMPLE 8

ELISA Assay with Fixed Cells

The cells to be studied for binding were suspended in PBS and adsorbed onto to a poly-L-lysine pretreated 96-well plate and kept at 4° C. The PBS was removed from the wells and an H₂O₂ solution in PBS (6% H₂O₂ in 200 μL/well) was added. The plate was incubated for 30 minutes at room temperature so as to inhibit the endogenous peroxidase that can interfere with the final substrate. The peroxide treatment was repeated, then the fixed cells were washed to remove the H2O2. A blocking buffer (1×PBS with 2% milk at 340 μL/well) was added to the plates, which were then incubated at 37° C. for 1 hour without agitation. The blocking buffer was removed.

M13 phage displaying one of the various polypeptides was added (1 μL) at a concentration of about 109 phage for each 200 μL well of incubation buffer (PBS/Ca/Mg/0.1% milk). The plates were incubated for 2 hours at 37° C. with agitation. The plate wells were washed 5 times with incubation buffer to reduce nonspecific binding of M13.

The binding of phage to the fixed cells was analyzed using anti-M13 antibody conjugated to horseradish peroxidase (“HRP/anti-M13”, 200 μL per well; HRP/anti-M13 monoclonal conjugate Pharmacia reference number 27-9421-01). After permitting the HRP/anti-M13 conjugate bind to any M13 bound to the fixed cells, the wells were washed 5 times with incubation buffer to remove unbound HRP/anti-M13 antibody conjugate. After washing, the HRP substrate TMB (200 μL/well; TMB for ELISA Sigma reference number T-8665) was added. The HRP reaction was permitted to progress at room temperature for 30 minutes before H₂SO₄ (100 μL per well) was added to stop the reaction. The HRP reaction products were analyzed, indicating the presence of M13 binding to cells, by observing the light absorbance at 450 nm.

### EXAMPLE 9

Template DNA Preparation for Sequencing

A. DNA Preparation for Sequencing of Bacteria Stored at 4° C.

A culture (2 mL LB/Amp/Tc) was inoculated with one colony of E. coli containing pSKAN phagemid and incubated at 37° C. overnight. The overnight culture was diluted with LB medium containing ampicillin (250 μg/mL), tetracycline (20 μg/mL) and helper phage M13KO7 (0.2 μL/mL of stock suspension at 10¹¹-10¹² cfu/mL). The culture was incubated at 37° C. without shaking for 15 minutes and then shaken for 1 hour until the culture is slightly turbid. Kanamycin (50 μg/mL) was added to the culture and agitated until OD was lower than 1. If the culture is permitted to become saturated, the bacteria may lyse and the ssDNA from M13pSKAN could be contaminated with bacterial genome and RF DNA. The culture was centrifuged in a sterile tube for 10 minutes at 8,000 rpm/min. The supernatant was transferred into a new tube and centrifugation repeated. The supernatant was sterile-filtered through a 0.45 μm filter to remove bacteria. The ssDNA was extracted from the phage using QIAprep® m13 from Qiagen.

B. DNA Preparation for Sequencing of Fresh Bacteria

A culture (10 mL LB/Amp/Tc) containing helper phage (0.2 μL/mL of a stock suspension at 10¹¹-10¹² cfu/mL) was inoculated with one colony of E. coli containing pSKAN phagemid. The culture was incubated at 37° C. for 1 hour until it was slightly turbid. Kanamycin (50 μg/mL) was added to the culture, which was then incubated with agitation until OD was higher but still under 1 (to prevent contamination of the ssDNA by bacterial genome and RF DNA). The culture was centrifuged in a sterile tube for 10 minutes at 8,000 rpm/min. The supernatant was transferred into a new tube and centrifuged again. The centrifuged supernatant was sterile-filtered through a 0.45 μm filter to remove all bacteria. The ssDNA was extracted from the phage using QIAprep® m13 from Qiagen.

C. Polymerase Chain Reaction (PCR)

The extracted DNA (100 ng) was subjected to PCR amplification using a set of primers surrounding the hypervariable oligonucleotide sequence coding for the peptide.

Primer 1 5′-GGGATTTTGCTAAACAAC-3′ SEQ ID NO:24

Primer 2 5′-GGAGGTCTAGATAACGAG-3′ SEQ ID NO:25

The reaction conditions were as follows: 25 mM MgCl₂, 0.3 mM dNTP, 5% glycerol, 5 units of Taq polymerase. The PCR cycle was repeated 30 times (94° C. for 30 seconds, 52° C. for 30 seconds, 72° C. for 30 seconds) with a final extended elongation at 72° C. for 10 minutes. The PCR products were then purified using QIA quick PCR purification kit (Qiagen).

### EXAMPLE 10

Effect of Carrier Molecule on the Differentiation of Bone Marrow AML Cells

A series of different peptides were displayed in phage and screened for cell binding specificity. The following table shows the effect of several of the best examples of peptides in bacteriophage on the differentiation of freshly obtained human bone marrow AML cells cultured for seven days in vitro.

| | Percent differentiated | Percent differentiated |
|---|---|---|
| Sample | in Specimen 1 | in Specimen 2 |
| Control | 7 | 27 |
| GM-CSF | 21 | 17 |
| Phage only | 30 | 35 |
| G5-12-8B | 28 | 61 |
| A2-11-24 | 79 | 50 |
| F7 12 8B | 38 | 33 |
| B4(1) | — | 53 |
| G2 11-24 | 16 | 39 |
| F1-12-8B | 34 | 52 |
| G2-11-8A | 28 | 51 |

### EXAMPLE 11

Cell-Specific Binding

Phage preparations were prepared displaying peptide inserts of eight amino acids in length. Several of the peptides were found to exhibit cellular specificity.

1. Phage displaying these peptides bind to CML and AML cells, but not to normal bone marrow, CD34+ cells or blood cells (lymphocytes or granulocytes)

| | | |
|---|---|---|
| G2 11-24 (tested on CD34+) | EFQQWSGK | SEQ ID NO:1 |
| B4(1) | DEKRGPNE | SEQ ID NO:16 |
| A2 11-24 | IEETARKG | SEQ ID NO:3 |
| E11 12-8B | VSEDIYDA | SEQ ID NO:22 |
| B6 12-8B | NHVCSRLG | SEQ ID NO:2 |
| G7 12-8B | NELHMKQH | SEQ ID NO:15 |

2. Phage displaying these peptides bind to CML, AML and CD34+ cells, but not to normal bone marrow or blood cells (lymphocytes or granulocytes).

| | | |
|---|---|---|
| A2 11-24 | IEETARKG | SEQ ID NO:3 |

3. This phage display preparation binds to CML and AML cells, but not to cord blood cells.

| | | |
|---|---|---|
| G2 11-24 | EFFQQWSK | SEQ ID NO:1 |
| F1 12-8B | VSEDIYDA | SEQ ID NO:22 |

4. These phage display preparations bind to CML and AML and cord blood cells.

| | | |
|---|---|---|
| E11 12-8B | VSEDIYDA | SEQ ID NO:22 |

5. Phage displaying these peptides bind to CLL, but not to normal peripheral blood cells (granulocytes and lymphocytes).

| | | |
|---|---|---|
| H3 11-24 | EFQQWSGK | SEQ ID NO:1 |
| C8 12-8B | NNATVEDE | SEQ ID NO:4 |

6. Phage displaying these peptides bind to all myeloid cells, both malignant and nonmalignant.

| | | |
|---|---|---|
| D2 11-24 | HSWKPDKL | SEQ ID NO:5 |
| C9 11-24 | NETTVREY | SEQ ID NO:19 |
| C10 12-8B | VSEDIYDA | SEQ ID NO:22 |
| G5 12-8B | EFQQWSGK | SEQ ID NO:1 |
| D9 12-8B | IEETARKG | SEQ ID NO:3 |
| G2 12-8A | NNATFEDG | SEQ ID NO:21 |
| D4 12-8A | ETGERIVL | SEQ ID NO:6 |
| F9 11-24 | NNATVEDE | SEQ ID NO:4 |
| B6 11-24 | NHVCSRLG | SEQ ID NO:2 |
| F10 11-24 | NETTVREY | SEQ ID NO:19 |
| F1 12-8B | VSEDIYDA | SEQ ID NO:22 |
| G6 12-8B | NELHMKQH | SEQ ID NO:15 |
| E2 12-8A | VSEDIYDA | SEQ ID NO:22 |

7. Phage displaying these peptides bind to CLL cells and normal peripheral blood cells (lymphocytes and granulocytes).

| | | |
|---|---|---|
| B4(2) | NHVCSRLG | SEQ ID NO:2 |
| D2 11-24 | HSWKPDKL | SEQ ID NO:5 |
| A7 11-24, C9 11-24 | NETTVREY | SEQ ID NO:19 |
| E5 12-8B | VSEDIYDA | SEQ ID NO:22 |
| G5 12-8B | EFQQWSGK | SEQ ID NO:1 |
| D9 12-8B | IEETARKG | SEQ ID NO:3 |
| G2 12-8A | NNATFEDG | SEQ ID NO:21 |
| D4 12-8A | ETGERIVL | SEQ ID NO:6 |

8. Phage displaying these peptides bind to CML and AML cells but not to CLL cells.

| | | |
|---|---|---|
| C8 12-8B | NNATVEDE | SEQ ID NO:4 |
| B7(1) | EFQQWSGK | SEQ ID NO:1 |
| F10 11-24 | NETTVREY | SEQ ID NO:19 |
| F1 12-8B | VSEDIYDA | SEQ ID NO:22 |
| E2 12-8A | VSEDIYDA | SEQ ID NO:22 |
| A2 11-24 | IEETARKG | SEQ ID NO:3 |
| B6 12-8B | NHVCSRLG | SEQ ID NO:2 |
| G7 12-8B | NELHMKQH | SEQ ID NO:15 |

9. Phage displaying these peptides bind to CML and AML cells and to CLL cells.

| | | |
|---|---|---|
| B4(2) | NHVCSRLG | SEQ ID NO:2 |
| D2 11-24 | HSWKPDKL | SEQ ID NO:5 |
| C9 11-24 | NETTVREY | SEQ ID NO:19 |
| C10 12-8B | VSEDIYDA | SEQ ID NO:22 |
| G5 12-8B | EFQQWSGK | SEQ ID NO:1 |
| D9 12-8B | IEETARKG | SEQ ID NO:3 |
| G2 12-8A | NNATFEDG | SEQ ID NO:21 |
| D4 12-8A | ETEGERIVL | SEQ ID NO:6 |
| C8 12-8B | NNATVEDE | SEQ ID NO:4 |

10. Phage displaying these peptides bind to normal bone marrow cells but not to AML cells.

| | | |
|---|---|---|
| G2 9-10-01 no Cys | CGREGEDW | SEQ ID NO:27 |
| G2 9-10-01 | CCGREGEDWC | SEQ ID NO:26 |
| B3 9-1-01 no Cys | KRGIHPES | SEQ ID NO:29 |
| B3 9-10-01 | CKRGIHPESC | SEQ ID NO:28 |
| F4 9-10-01 no Cys | QPTQYVMK | SEQ ID NO:30 |
| F4 9-10-01 | CQPTQYVMKC | SEQ ID NO:31 |

| | Peri. | cord | bone | | |
|---|---|---|---|---|---|
| | blood | blood | marrow | CD34+ | CD34− |
| | cells | cells | cells | cells | cells |
| 1 | − | NA | − | − | NA |
| 2 | − | NA | − | + | NA |
| 3 | NA | − | NA | NA | NA |
| 4 | NA | + | NA | NA | NA |
| 5 | − | NA | NA | NA | NA |
| 6 | + | + | + | + | + |
| 7 | + | NA | NA | NA | NA |
| 8 | NA | NA | NA | NA | NA |
| 9 | NA | NA | NA | NA | NA |
| 10 | NA | NA | NA | + | + |
| 11 | NA | NA | NA | + | − |

| | CLL cells | CML cells | AML cells |
|---|---|---|---|
| 1 | NA | + | + |
| 2 | NA | + | + |
| 3 | NA | + | + |
| 4 | NA | + | + |
| 5 | + | NA | NA |
| 6 | + | + | + |
| 7 | + | NA | NA |
| 8 | − | + | + |
| 9 | + | + | + |
| 10 | NA | NA | + |
| 11 | NA | NA | + |

### EXAMPLE 12

Effect of Carrier Molecule on the Differentiation of the Leukemic Cell Line HL60

This Example demonstrates that peptides with or without bacteriophage carriers can induce the differentiation of HL60 cells. In two cases, peptides with or without bacteriophage significantly inhibit the proliferation of the leukemia cells. Such inhibition is not the result of the killing of the cells. The data are graphed in the Figures.

FIG. 2 shows the leukemia cell numbers after 7 days of proliferation in the presence or absence of the different molecules. Studies were conducted of the effects of G5 12-8B peptide alone (artificially synthesized), G5 12-8B phage (the phage that is carrying the G5 12-8B peptide), the A2 11-24 peptide, the phage carrying the A2 11-24 peptide, the 2 peptides only, and the 2 phage.

The data graphed in FIG. 2 are also shown in the table below. The control number of cells per milliliter (2.6) is an average of results from dual controls (2.6×10⁶).

| | No. Cells (10⁶ | Percent relative to |
|---|---|---|
| Sample | cell/mL) | Controls |
| Cells | 2.6 | 100% |
| DMSO | 0.9 | 30% |
| G5 12-8B peptide | 1.2 | 48% |
| At 10⁻⁴ M | | |
| G5 12-8B phage 75 | 3.8 | 140% |
| A2 11-24 peptide | 2.0 | 77% |
| At 10⁻⁴ M | | |
| A2 11-24 phage 75 | 1.6 | 62% |
| Peptides A2 + G5 | 0.4 | 15% |
| Phages A2 + G5 | 1.4 | 54% |
| helper phage 75 | 1.8 | 67% |
| helper phage 150 | 1.65 | 63% |
| peptide G5 | 0.5 | 19% |
| at 3 × 10⁻⁴ M | | |
| peptide A2 | 1.8 | 69% |
| at 3 × 10⁻⁴ M | | |

The control experiments are (i) the cells themselves cultured in duplicate; (ii) DMSO-; and (iii) two different concentrations of phage which do not carry an artificial peptide. DMSO, dimethylsulfoxide, is a chemical that induces HL60 cellular differentiation.

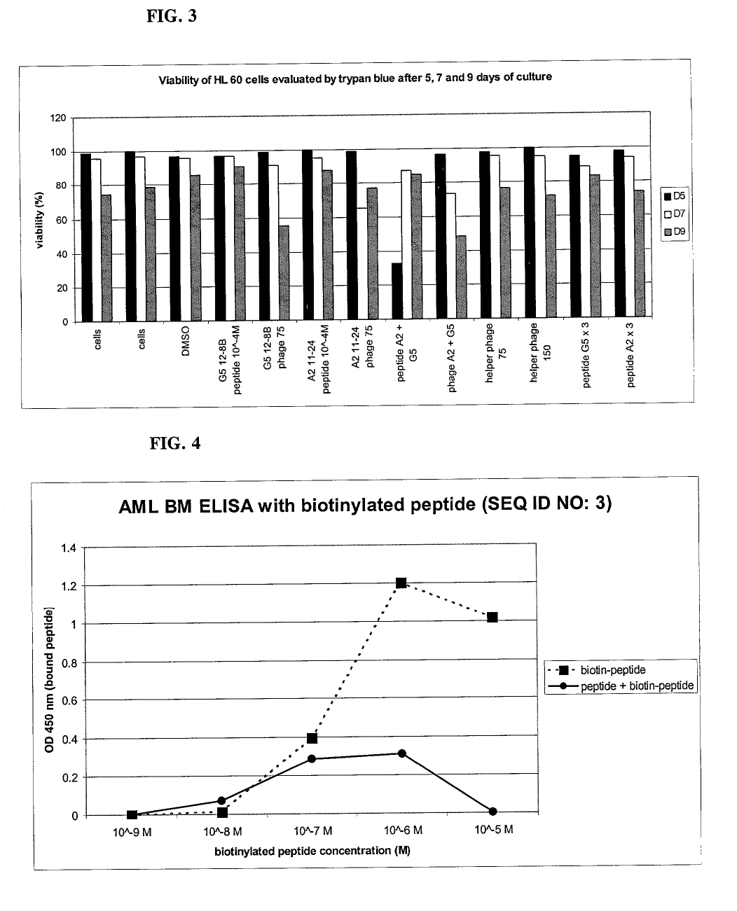

The data addressing the viability of cells grown under various conditions for 5, 7 or 9 days are shown in FIG. 3. Taking the viability data together with the proliferation data shown in FIG. 3, it was concluded that the presence of peptides A2 and G5 as well as the addition of G5 three times during the incubation result in a significant reduction in the number of cells present after 7 days of culture while the viability of the cells (FIG. 3) remained fairly good on day 7, demonstrating an inhibitory effect of the peptides on cell proliferation that is not based on the killing of the target cells.

The table below shows the data graphed in FIG. 3 for the viability of cells, in percent, on days 5, 7 and 9, using trypan blue detection of viable cells.

| | Day 5 | Day 7 | Day 9 |
|---|---|---|---|
| Sample | Viability | Viability | Viability |
| Cells | 100 | 96 | 76 |
| DMSO | 98 | 97 | 82 |
| G5 12-8B peptide | 98 | 96 | 90 |
| At 10⁻⁴ M | | | |
| G5 12-8B phage 75 | 99 | 97 | 57 |
| A2 11-24 peptide | 100 | 96 | 87 |
| At 10⁻⁴ M | | | |
| A2 11-24 phage 75 | 99 | 62 | 76 |
| Peptides A2 + G5 | 33 | 85 | 84 |
| Phages A2 + G5 | 98 | 74 | 47 |
| Helper phage 75 | 98 | 97 | 76 |
| Helper phage 150 | 100 | 96 | 71 |
| Peptide G5 | 96 | 90 | 84 |
| At 3 × 10⁻⁴ M | | | |
| Peptide A2 | 97 | 95 | 74 |
| At 3 × 10⁻⁴ M | | | |

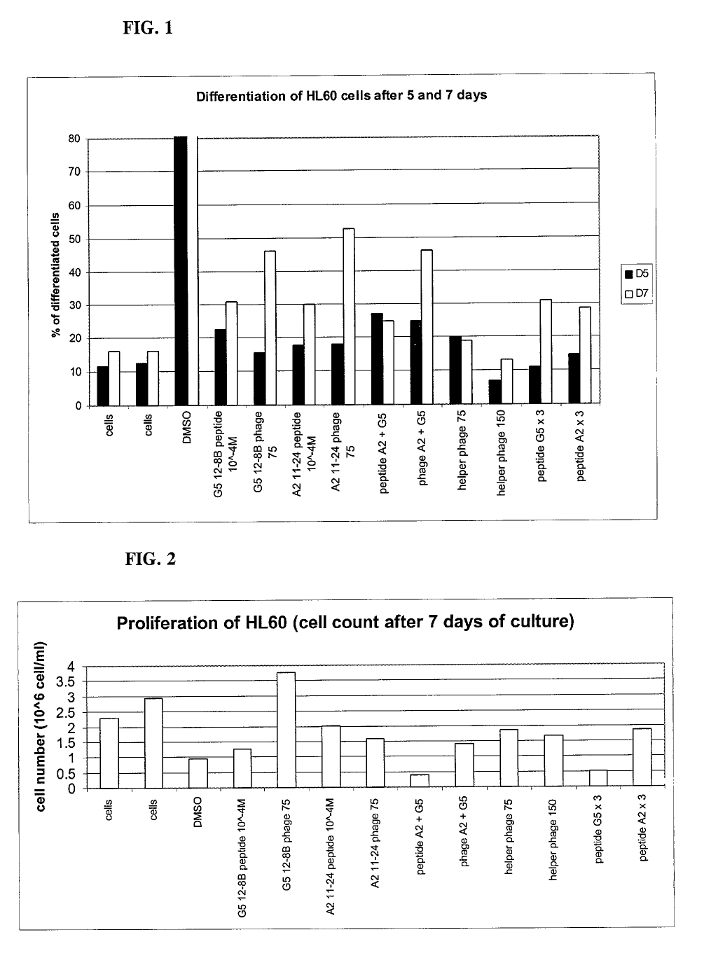

The level of differentiation of the HL60 cells after 5 or 7 days growth in the presence or absence of the various phage and peptides combinations described above are shown in FIG. 1. The high level of differentiation that has been achieved is notable from the graph in FIG. 1. The levels of differentiation in the presence of peptide exceed that produced by the presence of phage alone.

| | Day 5 | Day 7 | Day 9 |
|---|---|---|---|
| Sample | % Diff. | % Diff. | % Diff. |
| Cells | 12% | 16% | 16% |
| DMSO | off scale | off scale | off scale |
| G5 12-8B peptide | 22% (183%) | 31% (194%) | 22% |
| at 10⁻⁴ M | | | |
| G5 12-8B phage 75 | 17% (142%) | 46% (288%) | 46% |
| A2 11-24 peptide | 18% (150%) | 30% (188%) | 25% |
| at 10⁻⁴ M | | | |
| A2 11-24 phage 75 | 18% (150%) | 52% (325%) | 64% |
| peptides A2 + G5 | 27% (225%) | 25% (156%) | 27% |
| phages A2 + G5 | 25% (208%) | 46% (288%) | 76% |
| helper phage 75 | 20% (167%) | 19% (119%) | 21% |
| helper phage 150 | 7% (58%) | 13% (818%) | 26% |
| peptide G5 | 11% (92%) | 31% (194%) | 25% |
| at 3 × 10⁻⁴ M | | | |
| peptide A2 | 13% (108%) | 28% (175%) | 37% |
| at 3 × 10⁻⁴ M | | | |

### EXAMPLE 13

Effects on Cells from AML Patients

The effect of various phage-peptides were studied on the differentiation of cells from two AML patients. The results are shown in the table below. The number of cells are given in the millions; the percent differentiation shows the percentage of differentiated cells. “HP@” denotes phage that does not carry one of the experimental peptides.

| | Number of | | Percent |
|---|---|---|---|
| | Cells (106) | Viability | Differentiation |
| Patient 1 | | | |
| Control | 1.0 | 71% | 7% |
| GM-CSF | 1.57 | 80% | 21% |
| HP@ | 1.48 | 73% | 30% |
| G5-12-8B | 1.11 | 51% | 27.9% |
| A2-11-24 | 0.75 | 66% | 79.2% |
| F7-12-8B | 0.99 | 60% | 37.9% |
| B4(1) | 1.3 | 54% | ND |
| G2-11-24 | 1.11 | 62% | 16.2% |
| F1-12-8B | 0.82 | 54% | 34% |
| G2-12-8A | 0.93 | 52% | 27.5% |
| Patient 2 | | | |
| Control | 0.3 | 76% | 27% |
| GM-CSF | 0.87 | 89% | 16.5% |
| HP@ | 0.49 | 94% | 35% |
| GS-12-8B | 0.31 | 64% | 61% |
| A2-11-24 | 0.33 | 72% | 49.5% |
| F7-12-8B | 0.28 | 73% | 33% |
| B4(1) | 0.25 | 65% | 53% |
| G2-11-24 | 0.25 | 78% | 39% |
| F1-12-8B | 0.22 | 64% | 52% |
| G2-12-8A | 0.31 | 62% | 52% |

### EXAMPLE 14

Binding Studies for Peptides

The cell binding of the peptides to patient cells is evaluated by an ELISA assay using biotin-conjugated peptide. FIG. 4 shows that the binding intensity increased with the concentration of the peptide until a maximum binding concentration is obtained. The binding of the peptide can be competed by an excess of non-labeled peptide.

FIG. 4 shows that the binding of peptide A2 (SEQ ID NO:3) to patient bone marrow cells is saturable as shown by the total binding curve (squares with dotted line). The non-specific binding component is shown by competition of biotinylated peptide by binding with an excess of peptide (circles with solid line).

AML and normal bone marrow cells were stained with a conjugate of biotin with peptide A2 (biotin-peptide; 10⁻⁶M) and streptavidin-FITC. Competition of biotin-peptide is performed by the presence of a 100-fold concentration of peptide. Controls include cells by themselves, cells with streptavidin-FITC, and cells with biotin (10⁻⁶M) and streptavidin-FITC.

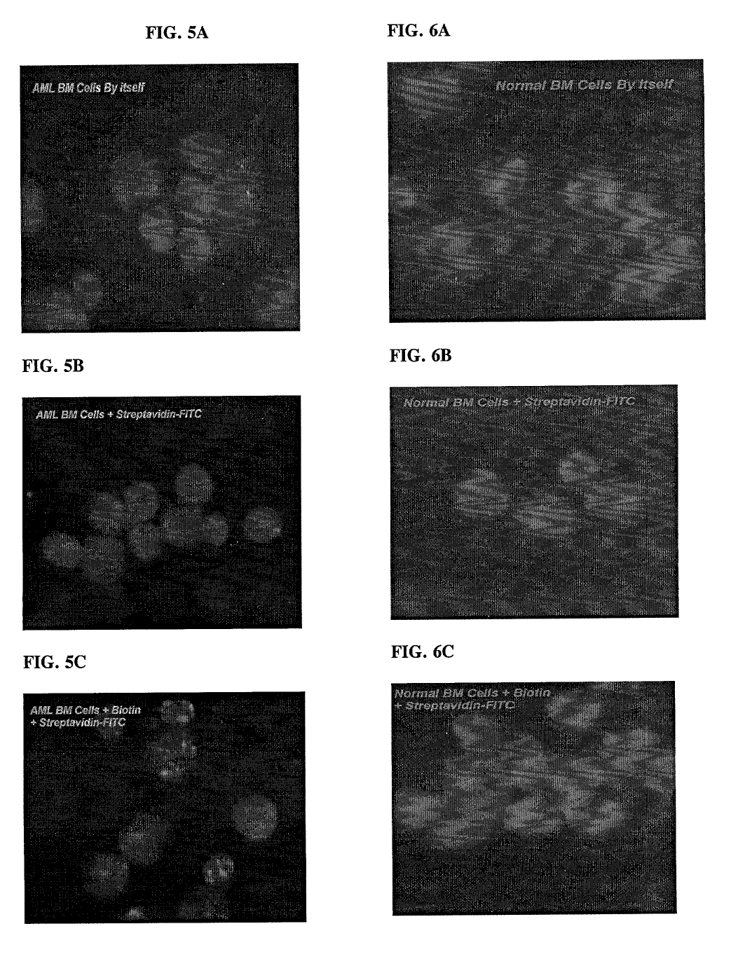

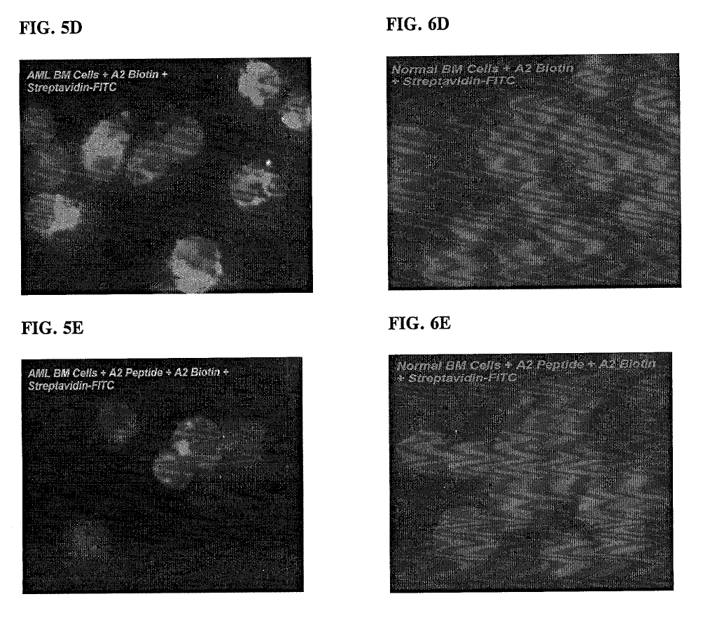

FIGS. 5 and 6 show the results of fluorescence binding studies performed that confirmed the leukemia-cell specific binding of peptide A2. Peptide A2 (SEQ ID NO:3) was visualized by fluorescence microscopy after incubation of the cells with biotin-peptide conjugate and streptavidin-FITC. FIG. 5 shows the distribution of fluorescence in bone marrow samples from a patient diagnosed with leukemia. FIG. 6 shows the distribution of fluorescence in bone marrow samples from a normal patient.

These biotinylated peptide and streptavidin-FITC examples demonstrate that the peptides are localized in the cytoplasm of AML bone marrow cells and that in an individual cell population, only some cells are labeled. The comparable examples with normal bone marrow cells as the targets demonstrate that the biotinylated peptide does not bind to normal cells. These studies use fixed cells, which may differ from leukemia cells in vivo.

Peptide A2 binds to a high degree in a specific manner to leukemic human cells. Some AML cells were clearly not stained with biotin-peptide, indicating that one or more sub-populations of cells express a target for the peptide. The negative control with biotin alone showed that the binding is via the peptide and not the biotin conjugate. In the competition control, the presence of an excess of the same peptide that was not conjugated to biotin decreased the intensity of the staining of the cells using biotin-peptide conjugate, thereby suggesting specific binding to the cells AML. Similar results of specific binding to subpopulations of cells were obtained with bone marrow samples from other patients diagnosed with leukemia.

Peptide A2 binds weakly in a non-specific manner to normal human cells. A slight signal was seen in normal bone marrow that was not competed out by an excess of peptide. This suggests that that weak signal is due to a non-specific interaction between the peptide and some other cellular targets on normal cells.

### EXAMPLE 15

Effects of Peptides on Proliferation and Cellular Differentiation

| Peptide | Sequence | SEQ ID NO: |
|---|---|---|
| A2 11-24 | CIEETARKGC | SEQ ID NO:7 |
| A2 11-24 no cys | IEETARKG | SEQ ID NO:3 |
| A2A | CIEETAAKGC | SEQ ID NO:8 |
| G5 12 8B | CEFQQWSGKC | SEQ ID NO:9 |
| G5 12-8B no cys | EFQQWSGK | SEQ ID NO:1 |
| B6 12 8B | CNHVCSRLGC | SEQ ID NO:10 |
| G7 12-8B | CNELHMKQHC | SEQ ID NO:11 |
| G2 12-8A | CNNATFEDGC | SEQ ID NO:12 |
| C8 12-8B | CNNATVEDEC | SEQ ID NO:13 |
| B4 (1) | CDEKRGPNEC | SEQ ID NO:14 |

| | Activation of | | Inhibition | |
|---|---|---|---|---|
| | Proliferation | | of | |
| Peptide | (Patient No.) | | proliferation | |
| | | +GM-CSE | | +GM-CSF |
| A2 11-24 | 2, 3, 4, 5, 9 | 3, 4, 12 | | |
| A2 11-24 no cys | 3, 5, 6, 9 | 11 | | |
| A2A | 3, 4, 6, 9, | 3, 4 | 10 | |
| G5 12 8B | 3, 9, 12 | 3, 11 | | |
| G5 12-8B no cys | 2, 3, 6, 9 | 3, 12 | 1, 10 | |
| B6 12 8B | 2, 3, 4, 5, 9 | 2, 3, 11 | 1, 10, 12 | |
| G7 12-8B | 9, 14 | | | |
| G2 12-8A | 1, 2, 3, 5, 6, | 3, 2 | 10 | |
| | 9 | | | |
| C8 12-8B | 5, 6 | 11 | | |
| B4 (1) | 9 | 11 | | |

| Peptide | Activation of Differentiation | | SEQ ID NO: |
|---|---|---|---|
| A2 11-24 | 2, 12, 14 | 3 | 7 |
| A2 11-24 no cys | 1, 2, 6, 14 | 3, 11 | 3 |
| A2A | 7, 10, 14 | 3 | 8 |
| G5 12 8B | 11, 14 | 3, 11 | 9 |
| G5 12-8B no cys | 1, 2, 11, 14 | 3 | 1 |
| B6 12 8B | 1, 11, 14 | 3, 4 | 2 |
| G7 12-8B | 10, 11, 12, 14 | | 15 |
| G2 12-8A | 2, 14 | 11, 3 | 21 |
| C8 12-8B | 12 | 11 | 13 |
| B4 (1) | 12, 14 | | 16 |

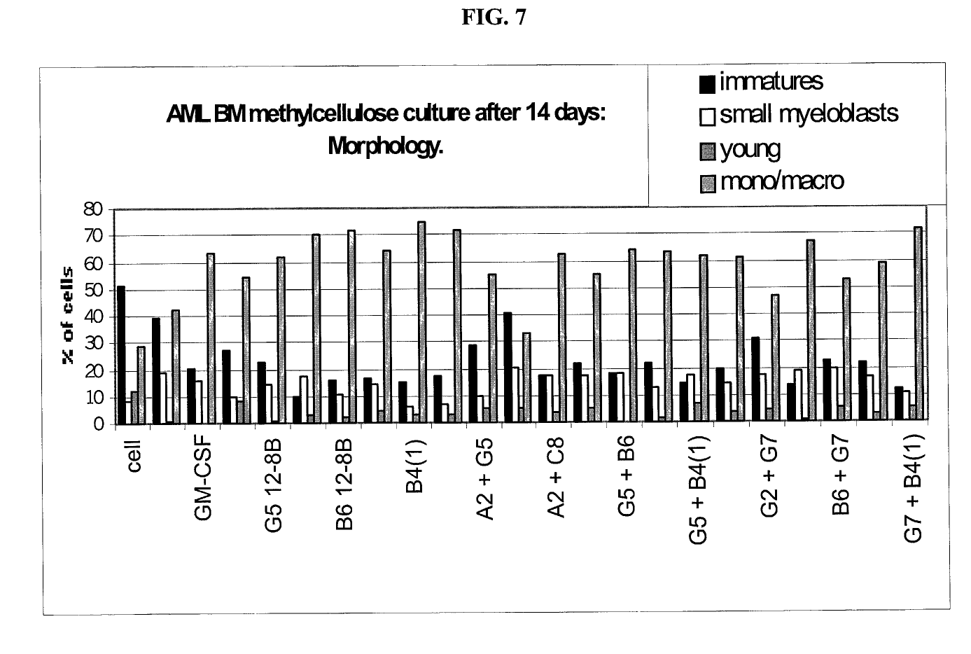

The effect of various peptides were analyzed for their biological activity on cells from a patient with acute myelogenous leukemia. The AML cells were cultured into a semi-solid medium of methylcellulose in order to determine the cloning efficiency and the differentiation of the cells. The various peptides were introduced into the semi-solid cell culture at a concentration of 10⁻⁴ M (0.1 mM) peptide. After 14 days continuous incubation of the cell cultures, the morphology of the cells were analyzed. The data are shown in FIG. 7 for the cultures and show the percentage of immature cells in the culture (solid black), the percentage of small myeloblasts (solid white), the percentage of young cells (dark gray), and the relative percentage of monocytes to macrocytes (light gray). The data regarding the biological response of AML bone marrow cells in the presence of the various peptides is shown in the table below.

| | Activation of | | Inhibition of | | Cell | |
|---|---|---|---|---|---|---|
| Peptide | Proliferation | | Proliferation | | Differentiation | |
| SEQ ID NO: | | GM-CSF | | GM-CSF | | GM-CSF |
| A2 | 5/11 | 3/4 | | | 3/9 | 1/4 |
| SEQ ID NO:7 | | | | | | |
| A2 | 4/14 | 1/2 | | | 4/13 | 2/2 |
| no Cys | | | | | | |
| SEQ ID NO:3 | | | | | | |
| A2A (mut. A2) | 4/11 | 2/4 | 1/11 | | 3/8 | 1/4 |
| SEQ ID NO:8 | | | | | | |
| G5 | 3/8 | 2/3 | | | 2/6 | 1/3 |
| SEQ ID NO:1 | | | | | | |
| B6 | 5/14 | 3/5 | 3/14 | | 3/13 | 2/5 |
| SEQ ID NO:2 | | | | | | |
| G7 | 2/6 | 0/2 | | | 4/5 | 0/2 |
| SEQ ID NO:15 | | | | | | |
| G2 | 6/14 | 2/5 | 1/14 | | 2/12 | 2/5 |
| SEQ ID NO:21 | | | | | | |
| C8 | 2/8 | 1/2 | | | 1/8 | 1/2 |
| SEQ ID NO:4 | | | | | | |
| B4 (1) | 1/6 | 1/2 | | | 2/4 | 0/2 |
| SEQ ID NO:16 | | | | | | |

The combination of peptide with GM-CSF modulated the activity of GM-CSF. The effects of the peptide with GM-CSF was in some cases additive with respect to the colony numbers. Apparently, the intracellular pathways were activated by both GM-CSF and peptide amplified the biological response. In some cases, with respect to cellular differentiation, GM-CSF was found to inhibit or to amplify the activity of the studied peptide.

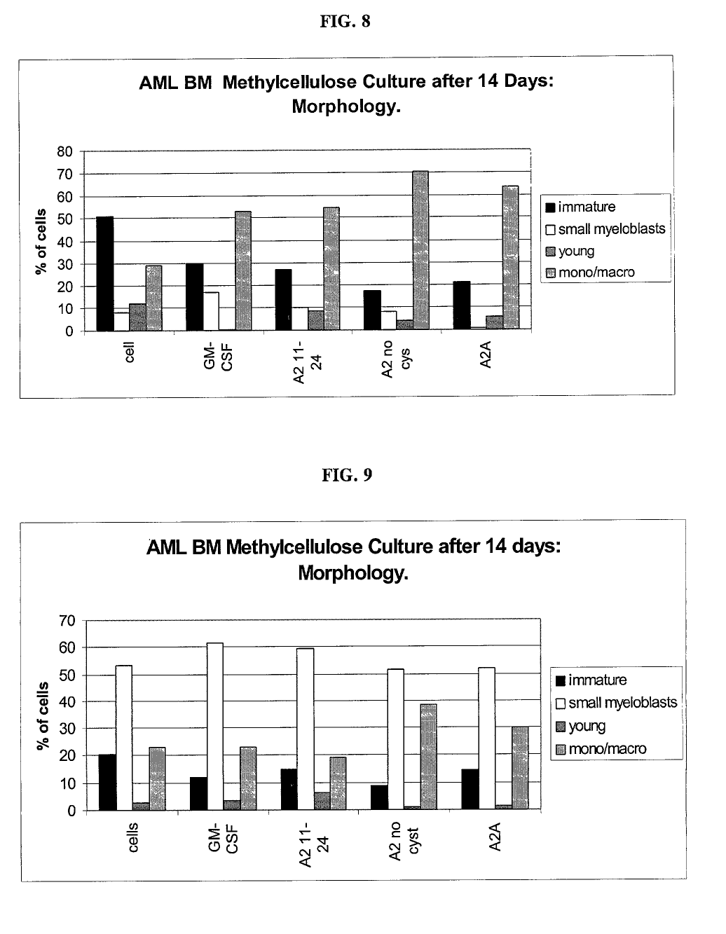

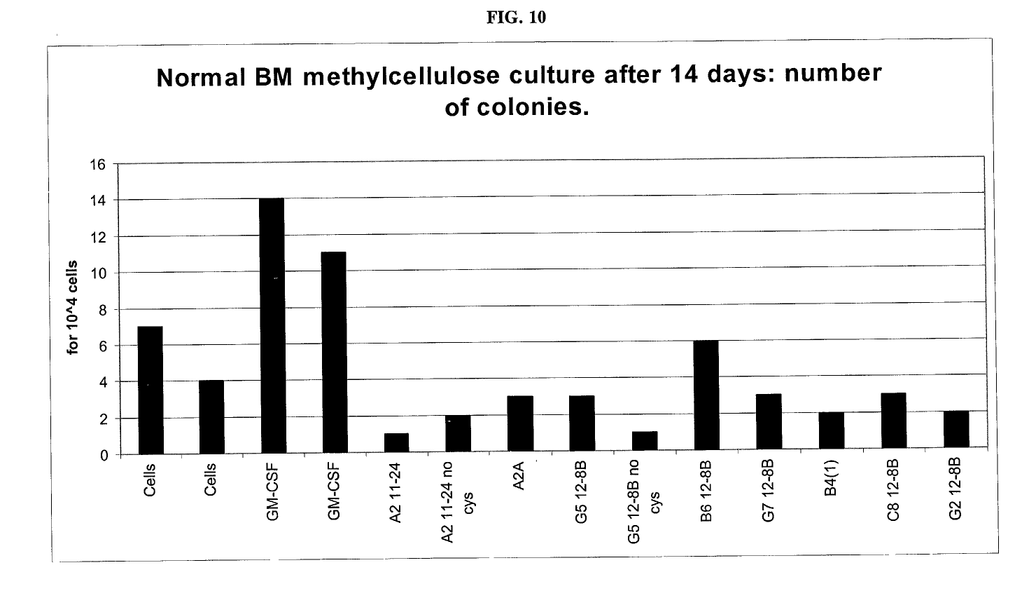

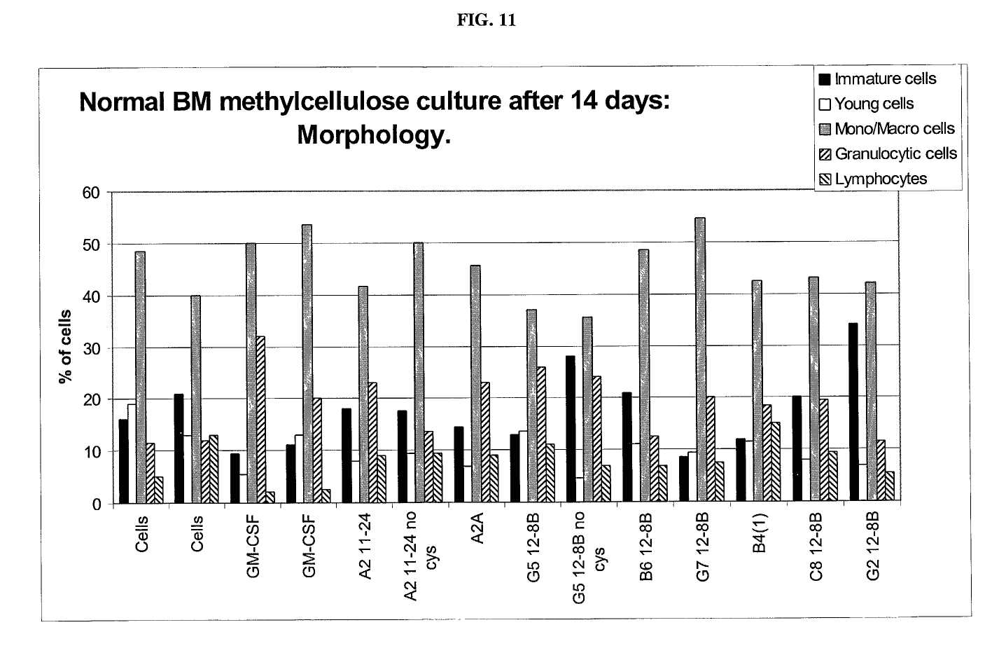

The peptide (A2) having SEQ ID NO:3 was tested with some different variations: peptide surrounded (A2) or not (A2 no Cys) by two terminal cysteine residues, and the peptide with one mutation (R replaced by A=A2A). FIG. 8 shows that the percentage of immature cells decreased while the percentage of differentiated cells such as macrophages increased showing that the three variations of the peptide was able to induce the differentiation of the patient cells. The activity of the peptide not flanked by cysteine residues (A2 no Cys) and the mutated peptide (A2A) showed a higher activity on the cell differentiation that the peptide with cysteine residues. FIG. 9 shows an example where the peptide with the cysteine residues and the mutated form have a biological activity while the peptide with the cysteine residues doesn't have any activity on cell differentiation. This indicates that the conformation of the peptide plays an important role in its activity.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, culture media, culture media supplements, chromatic or fluorescent dyes for staining or labeling a specific targets, radioactive isotopes for labeling specific targets, and/or disposable lab equipment, such as multi-well plates in order to readily facilitate implementation of the present methods.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims.

What is claimed is:

1. An isolated polypeptide comprising a sequence selected from one of:
(a) SEQ ID NOS:1-23;
(b) SEQ ID NOS:26-31;
a (c) SEQ ID NOS:1-23 having one or more conservative amino acid substitutions; or
(d) SEQ ID NOS:26-31 having one or more conservative amino acid substitutions.

2. The isolated polypeptide of claim 1 wherein the sequence is selected from one of (A) or (B).

3. The isolated polypeptide of claim 1 wherein the sequence is selected from SEQ ID NO:1 or SEQ ID NO:9.

4. The isolated polypeptide of claim 1 wherein the sequence is selected from SEQ ID NO:2 or SEQ ID NO:10.

5. The isolated polypeptide of claim 1 wherein the sequence is selected from SEQ ID NO:3 or SEQ ID NO:7.

6. The isolated polypeptide of claim 1 wherein the sequence is selected from SEQ ID NO:8.

7. The isolated polypeptide of claim 1 wherein the sequence is selected from SEQ ID NO:4 or SEQ ID NO:13.

8. The isolated polypeptide of claim 1 wherein the sequence is selected from SEQ ID NO:5 or SEQ ID NO:17.

9. The isolated polypeptide of claim 1 wherein the sequence is selected from SEQ ID NO:6 or SEQ ID NO:18.

10. The isolated polypeptide of claim 1 wherein the sequence is selected from SEQ ID NO:12 or SEQ ID NO:21.

11. The isolated polypeptide of claim 1 wherein the sequence is selected from SEQ ID NO:11 or SEQ ID NO:15.

12. The isolated polypeptide of claim 1 wherein the sequence is selected from SEQ ID NO:14 or SEQ ID NO:16.

13. The isolated polypeptide of claim 1 wherein the sequence is selected from SEQ ID NO:19 or SEQ ID NO:20.

14. The isolated polypeptide of claim 1 wherein the sequence is selected from SEQ ID NO:22 or SEQ ID NO:23.

15. The isolated polypeptide of claim 1 wherein the sequence is selected from SEQ ID NO:26 or SEQ ID NO:27.

16. The isolated polypeptide of claim 1 wherein the sequence is selected from SEQ ID NO:28 or SEQ ID NO:29.

17. The isolated polypeptide of claim 1 wherein the sequence is selected from SEQ ID NO:30 or SEQ ID NO:31.

18. The isolated polypeptide of claim 2 wherein the polypeptide comprises part of a carrier protein.

19. The isolated polypeptide of claim 2 further comprising an accessory molecule.

20. The isolated polypeptide of claim 19 wherein the accessory molecule is a tag molecule, identification molecule, chemotherapeutic agent, radiopharmaceutical, cytotoxic agent, treatment molecule, antigenic molecule, antibody fragment or antibody.

21. The isolated polypeptide of claim 1 wherein the polypeptide consists essentially of a sequence selected from (A), (B), (C) or (D).

22. A method for binding a peptide to a cell, comprising:
contacting a polypeptide comprising a sequence selected from one of:
(a) SEQ ID NOS:1-23;
(b) SEQ ID NOS:26-31;
(d) SEQ ID NOS:1-23 having one or more conservative amino acid substitutions; or
(d) SEQ ID NOS:26-31 having one or more conservative amino acid substitutions,
with one or more cells for a period of time sufficient to produce one or more polypeptide-bound cells.

23. The method of claim 22 wherein the polypeptide comprises a sequence selected from (a) or (b).

24. The method of claim 22 further comprising separating any polypeptide-bound cells.

25. The method of claim 22 wherein the one or more cells are leukemia cells.

26. The method of claim 23 wherein the polypeptide is attached to a solid support or a viral envelope.

27. The method of claim 23 further comprising measuring the number of polypeptide-bound cells.

28. The method of claim 22 wherein the polypeptide comprises part of a carrier protein.

29. The method of claim 22 wherein the polypeptide comprises an accessory molecule.

30. The method of claim 22 wherein the accessory molecule is a tag molecule, detection molecule, chemotherapeutic agent, radiopharmaceutical, cytotoxic agent, treatment molecule, antigenic molecule, antibody fragment or antibody.

31. The method of claim 22 wherein the polypeptide consists essentially of a sequence selected from one of (a), (b), (c) or (d).

32. A kit for binding polypeptides to cells comprising instructions for carrying out the method of claim 22.

33. The kit of claim 32 further comprising one or more polypeptides comprising a sequence selected from one of:
(a) SEQ ID NOS:1-23;
(b) SEQ ID NOS:26-31;
(c) SEQ ID NOS:1-23 having one or more conservative amino acid substitutions; or
(d) SEQ ID NOS:26-31 having one or more conservative amino acid substitutions.

34. A method of inducing an acute myelogenous leukemia cell differentiation into a mature hematopoietic cell comprising:
(a) contacting an acute myelogenous leukemia cell with a peptide that preferentially binds to the acute myelogenous leukemia cell to produce a peptide-bound acute myelogenous leukemia cell; and
(b) inducing differentiation of the peptide-bound acute myelogenous leukemia cell into a mature blood cell that performs normal blood cell function.

35. The method of claim 34 wherein the peptide induces differentiation of the peptide-bound acute myelogenous leukemia cell into the mature blood cell.

36. The method of claim 34 wherein the peptide is bound to a treatment molecule which induces differentiation of the peptide-bound acute myelogenous leukemia cell into the mature blood cell.

37. The method of claim 34 wherein steps (a) and (b) are performed in vivo.

38. The method of claim 37 further comprising contacting one or more additional acute myelogenous leukemia cells with a chemotherapeutic agent.

39. An isolated peptide comprising a binding region which binds preferentially to a surface of a blood cell with the proviso that the peptide does not bind to an immunogenic molecule on the surface of the blood cell.

40. The isolated peptide of claim 39 wherein the blood cell is a leukemia cell.

41. The isolated polypeptide of claim 39 wherein the polypeptide comprises part of a carrier protein.

42. The isolated polypeptide of claim 39 further comprising an accessory molecule.

43. The isolated polypeptide of claim 39 wherein the accessory molecule is a tag molecule, identification molecule, chemotherapeutic agent, radiopharmaceutical, cytotoxic agent, treatment molecule, antigenic molecule, antibody fragment or antibody.

44. The isolated polypeptide of claim 40 wherein the polypeptide induces differentiation of the leukemia cell into a mature blood cell capable of normal blood cell function.

45. The isolated polypeptide of claim 40 wherein the polypeptide inhibits proliferation of the leukemia cell.

46. A method for specifically binding a peptide with a cellular component comprising contacting a portion of a cell membrane of a hematopoietic cell with a peptide that preferentially binds to a component of the cell membrane of the hematopoietic cell for a sufficient period of time to allow the peptide to bind to the component of the cell membrane, wherein the component of the cell membrane that is preferentially bound by the peptide is weakly immunogenic or not immunogenic.

47. The method of claim 46 further comprising separating the portion of the hematopoietic cell membrane bound to the peptide.

48. The method of claim 46 wherein the peptide is bound to an accessory molecule or a solid support.

49. The method of claim 48 wherein the accessory molecule is a tag molecule, identification molecule, chemotherapeutic agent, radiopharmaceutical, cytotoxic agent, treatment molecule, antigenic molecule, antibody fragment or antibody.

50. The method of claim 47 wherein the hematopoietic cell is a leukemia or a preleukemia cell.

51. The method of claim 47 further comprising identifying the portion of the hematopoietic cell membrane bound to the peptide.
